(19) United States
(12) Patent Application Publication
(10) Pub. No.: US 2021/0070731 A1
(43) Pub. Date: Mar. 11, 2021

(54) TRICYCLIC KINASE INHIBITORS AND USE THEREOF

(71) Applicants: Nivedita Namdev, Westford, MA (US); Reena Zutshi, Tucson, AZ (US)

(72) Inventors: Nivedita Namdev, Westford, MA (US); Reena Zutshi, Tucson, AZ (US)

(21) Appl. No.: 17/012,788

(22) Filed: Sep. 4, 2020

Related U.S. Application Data
(60) Provisional application No. 62/896,269, filed on Sep. 5, 2019.

(51) Int. Cl. C07D 401/04

(52) U.S. Cl. CPC C07D 401/04

(57) ABSTRACT

The present application provides novel compounds that are inhibitors of kinases, including AMPK-related kinases like NUAK1, NUAK2, SIK1, SIK2, SIK3, MARK1, MARK2, MARK3, MARK4, as well as AURKA, AURKB, AURKC, CLK1, CLK2, DCAMKL2, MAPK7, MKNK2, PIK3CD, PKN3, RET, TAOK1, TAOK2, TAOK3, ULK2 and their mutants. The application also provides compositions, including pharmaceutical compositions, kits that include compounds, and methods of making and using compounds. The compounds provided herein are useful in treating diseases, disorders, or conditions that are mediated by these kinases.

### CROSS-REFERENCES TO RELATED APPLICATIONS

This application is non-provisional and claims benefit of U.S. Patent Application No. 62/896,269, filed Sep. 5, 2019, the specification(s) of which is/are incorporated herein in their entirety by reference.

### FIELD

The present application provides novel tricyclic pyrido-diazepine compounds that are inhibitors of protein kinases and the use of such compounds for treatment of diseases, disorders and conditions.

### BACKGROUND

Kinases are a large superfamily of structurally related proteins, with almost all kinases containing a conserved 250-300 amino acid “kinase domain”, which imparts catalytic activity to the protein. The kinase superfamily is comprised of over 500 members, constituting 1.7% of the human genome. Kinases catalyze the transfer of a γ-phosphoryl group from a nucleotide triphosphate to a hydroxyl group of their substrate protein/peptide, and in doing so mediate signal transduction within a cell.

The kinase domain appears in a number of polypeptides, including transmembrane receptors, intracellular receptor associated polypeptides, cytoplasmic located polypeptides, nuclear located polypeptides and subcellular located polypeptides. Kinases may be regulated by one or more mechanisms. These mechanisms include, for example, autophosphorylation, transphosphorylation by other kinases, protein-protein interactions, protein-lipid interactions, protein-polynucleotide interactions, ligand binding, and post-translational modification.

The kinase domain comprises two lobes can be further subdivided into twelve subdomains. The N-terminal portion forms the small lobe (including Subdomains I-IV) whose architecture is composed of an antiparallel five-strand b-sheet and one a-helix, while the lower C-terminal domain forms another lobe (including Subdomains VIA-XI) containing mostly helical architecture. Subdomain V spans the two lobes. The N-terminal domain is thought to participate in orienting the nucleotide (or other binding entity), while the C-terminal domain is thought to be responsible for binding peptide substrate and initiating phosphotransfer to the hydroxyl group of a serine, threonine, or tyrosine residue. The N- and C-terminal domains are connected through a single peptide strand, to which the adenine moiety of ATP and/or GTP binds via an eleven membered hydrogen bond cycle, involving the N1 and the N6 amino group, and the backbone carbonyl and NH functions of two nonconsecutive residues. This linker acts as a hinge about which the domains can rotate with respect to each other without disruption of the secondary architecture of the kinase. Several torsion angle changes in the linker backbone allow this movement to occur. The ribose group of ATP is anchored to the enzyme via hydrogen bonds with residues within the ribose-binding pocket. The triphosphate group is held in position via various polar interactions with several variable residues from the glycine rich loop, the conserved DFG motif and the catalytic loop.

Kinases can be classified based on the substrates they phosphorylate (for example, serine/threonine kinases predominantly phosphorylate substrate on serine and/or threonine residues, tyrosine kinases phosphorylate substrates on tyrosine residues, dual-specificity kinases phosphorylate substrates on tyrosine, serine and/or threonine residues, lipid kinases phosphorylate lipids). Phosphorylation events catalyzed by kinases act as molecular on/off switches that can modulate or regulate the biological function of the target protein. Phosphorylation of target proteins occurs in response to a variety of extracellular signals, for example, hormones, neurotransmitters, growth and differentiation factors, etc., cell cycle events, environmental or nutritional stresses, etc. Protein and lipid kinases can function in signaling pathways to activate or inactivate, or modulate, either directly or indirectly, the activity of their target substrates, which may include, for example, metabolic enzymes, regulatory proteins, receptors, cytoskeletal proteins, ion channels or pumps, or transcription factors. Phosphorylation, therefore, controls and regulates many cellular processes, including, but not limited to, metabolism, transcription, translation, cell cycle, cell motility, apoptosis, cell differentiation, proliferation, intracellular communication, homeostasis and functioning of nervous and immune systems. Uncontrolled signaling due to defective control of protein phosphorylation has been implicated in a number of diseases and disease conditions, including, for example, inflammation, cancer, allergy/asthma, disease and conditions of the immune system, disease and conditions of the central nervous system (CNS), cardiovascular disease, dermatology, and angiogenesis.

AMPK (5′ adenosine monophosphate-activated protein kinase) is a serine/threonine kinase that has been shown to regulate cellular growth, metabolism and energy homeostasis. Recently, several kinases have been identified that share sequence homology with the catalytic alpha-subunit of AMPK and are activated by phosphorylation of a conserved threonine in the T-loop of the kinase by LKB1. These 12 kinases, also known as AMPK-related kinases, include BRSK1, BRSK2, NUAK1, NUAK2, SIK1, SIK2, SIK3, MARK1, MARK2, MARK3, MARK4 and MELK. The AMPK-related kinases regulate a multitude of cell functions; NUAK kinases modulate cell adhesion, cancer cell invasion, microtubule stability, antioxidant stress response, embryonic development, senescence, proliferation, neuronal polarity and axon branching; BRSKs and MARKs have been shown to regulate cell polarity; and SIKs mediate gene transcription immunoregulatory functions of cells.

NUAK1 has been shown to phosphorylate tau and is a critical regulator of tau metabolism and toxicity, which contributes to Alzheimer's and other neurodegenerative diseases, together known as tauopathies. NUAK1 has been characterized in post-mortem brains of patients with Alzheimer's disease and Progressive Supranuclear Palsy, colocalized with tau neurofibrillary tangles as well as tan neuropril threads. NUAK1 is a target of several miRNAs that are frequently suppressed in cancer suggesting a role for NUAK1 in tumorigenesis. NUAK1 has been shown to sustain viability of cancer cells in MYC-driven cancers. Overexpression of NUAK1 is associated with poor prognosis in multiple cancers, including breast, colorectal, ovarian, lung, liver and others.

SIK (Salt-inducible kinases) subfamily comprises 3 members, SIK1, SIK2 and SIK3, SIKs are involved in modulation of toll-like receptor (TLR)-induced pro-inflammatory signals. In macrophages, SIKs limit the production of anti-inflammatory cytokines (for example IL-10) and inhibition of SIK pharmacologically is associated with decrease in pro-inflammatory cytokines. SIKs regulate various metabolic pathways involved in glucose and lipid homeostasis, deregulation of which contributes to the development of metabolic disorders like type-2 diabetes. SIKs are overexpressed in several cancers and inhibition of SIK2 in cancer cells is associated with significant decrease in cancer cell growth and proliferation.

The development of selective protein kinase inhibitors that can block the disease pathologies and/or symptoms resulting from aberrant protein kinase activity has therefore generated much interest. Disclosed herein are compounds with inhibitory activity against AMPK-related kinases like NUAK1, NUAK2, SIK1, SIK2. SIK3, MARK1, MARK2, MARK3, MARK4, as well as AURKA, AURKB, AURKC, CLK1, CLK2, DCAMKL2, MAPK7, MKNK2, PIK3CD, PKN3, RET, TAOK1, TAK2, TAOK3, ULK2 and their mutants. Also disclosed are methods for preparing the compounds and pharmaceutical compositions containing them. In addition, methods are disclosed for treatment of diseases mediated by any of these kinases, including cases that are resistant to known treatments of care.

### SUMMARY

The present application provides novel compounds that are inhibitors of kinases, including AMPK-related kinases like NUAK1, NUAK2, SIK1, SIK2, SIK3, MARK1, MARK2, MARK3, MARK4, as well as AURKA, AURKB, AURKC, CLK1, CLK2, DCAMKL2, MAPK7, MKNK2, PIK3CD, PKN3, RET, TAOK1, TAOK2, TAOK3, ULK2 and their mutants. The application also provides compositions, including pharmaceutical compositions, kits that include compounds, and methods of making and using compounds. The compounds provided herein are useful in treating diseases, disorders, or conditions that are mediated by these kinases.

In some embodiments, the present disclosure provides compounds represented by structural Formula 1 and 1A:

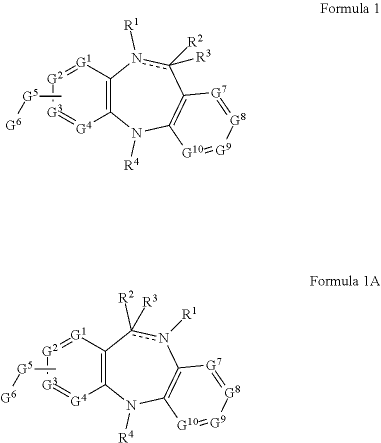

or a pharmaceutically acceptable salt thereof;
wherein:
Each G¹, G², G³, G⁴, G⁷, G⁸, G⁹ and G¹⁰ is independently selected from N and CR¹⁰;
G is selected from —S—, —O—, —NR^{a}—, —C(O)—, —C(O)O—, C(O)NR^{a}—, S(O)NR—, —S(O)₂NR—, —S(O)—, —S(O)₂—, C₁₋₆ alkylene, C₂₋₆ alkenylene and C₂₋₆ alkynylene; wherein each C₁₋₆alkylene, C₂₋₆ alkenylene and C₂₋₆alkynylene is substituted with one to six R¹⁰⁰;
G⁶ is selected from C₄₋₇ heterocycloalkyl, C₆₋₁₀ aryl, 5-10 membered heteroaryl containing 1 to 4 heteroatoms selected from the group consisting of N, O, and S and 4-10 membered heterocyclyl containing 1 to 4 heteroatoms selected from the group consisting of N, O, and S; wherein each C₆₋₁₀ aryl, 5-10 membered heteroaryl and 4-10 membered heterocyclyl is optionally substituted with one to four R¹⁰⁰;
R¹ is selected from absent, hydrogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{g}, —S(O)₂R^{g}, —NR^{a}R^{b}, —OR^{a}, —SR^{b}, C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocycyl containing 1 to 4 heteroatoms selected from the group consisting of N, O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocyclyl is optionally substituted with one to four R¹⁰⁰;
R² is selected from absent, hydrogen, halogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{b}, —S(O)₂R^{g}, —NR^{a}R^{b}, —OR^{a}, —SR^{b}, C₁₋₆ alkyl, C₂₋₆alkenyl, C₂₋₆alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocyclyl is optionally substituted with one to four R¹⁰⁰;
R³ is selected from hydrogen, halogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{g}, —S(O)₂R^{g}, —NR^{a}R^{b}, —OR^{a}, —SR^{b}, C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N. O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocyclyl is optionally substituted with one to four R¹⁰⁰; and optionally R² and R³ together with the carbon atom to which it is attached may form a group selected from —C(O) and —C═CR^{a}R^{b};
R⁴ is selected from hydrogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{g}, —S(O)₂R^{g}, —NR^{a}R^{b}, —OR^{a}, —SR^{b}, C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆ alkyl, C₂₋₆ alkenyl, C₂₋₆alkynyl, C₃₋₈ cycloalkyl, C₆₋₁₀aryl, 5-10 membered heteroaryl and 4-10 membered heterocyclyl is optionally substituted with one to four R¹⁰⁰;
Each R¹⁰ is independently selected from hydrogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{g}, —S(O)₂R^{g}, —NR^{a}R^{b}, —OR^{a}, —SR^{b}, C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆ alkyl, C₂₋₆ alkenyl, C₂₋₆ alkynyl, C₃₋₈ cycloalky, C₆₋₁₀ aryl, 5-10 membered heteroaryl and 4-10 membered heterocyclyl is optionally substituted with one to four R¹⁰⁰;
Each R^{a} and R^{b} is independently selected from hydrogen, C1-6 alkyl, C2-6 alkenyl and C2-6 alkynyl; wherein each C1-6 alkyl, C2-6 alkenyl, C2-6 alkynyl, is optionally substituted with one to four R²⁰⁰;
Each R^{c} and R^{d} is independently selected from hydrogen, C6-10 aryl, C1-6 alkyl, C2-6 alkenyl and C2-6 alkynyl;
Each R^{e} and R^{f} is independently selected from hydrogen, C1-6 alkyl, C2-6 alkenyl and C2-6 alkynyl;
Each R^{g} is independently selected from C₁₋₆ alkyl, C₂₋₆ alkenyl, C₂₋₆ alkynyl, C3-8 cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆alkyl, C₂₋₆alkenyl, C₂₋₆alkynyl, C₃₋₈ cycloalkyl, C₆₋₁₀ aryl, 5-10 membered heteroaryl and 4-10 membered heterocyclyl is optionally substituted with one to four R²⁰⁰; each R¹⁰⁰ is independently selected from hydrogen, halogen, cyano, hydroxy, amino, oxo, thioxo, vinyl, —C(O)R^{c}, —C(O)OR^{c}, —C(O)NR^{c}R^{d}, —N(R^{c})C(O)R^{d}, —S(O)NR^{c}R^{d}, —S(O)₂NR^{c}R^{d}, —S(O)R^{c}, —S(O)₂R^{c}, —NR^{c}R^{d}, —OR^{c}, —SR^{c}, C₁₋₆ alkyl, C₂₋₆ alkenyl and C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆ alkyl, C₂₋₆ alkenyl, C₂₋₆ alkynyl, C₃₋₈ cycloakyl, C₆₋₁₀ aryl, 5-10 membered heteroaryl and 4-10 membered heterocyclyl is optionally substituted with one to four R²⁰¹; each R^{e} and R^{f} is independently selected from hydrogen, C₁₋₆ alkyl, C₂₋₆ alkenyl and C₂₋₆ alkynyl; each R²⁰¹ is independently selected from hydrogen, halogen, cyano, hydroxy, amino, oxo, thioxo, vinyl, —C(O)R^{e}, —C(O)OR^{e}, —C(O)NR^{e}R^{f}, —N(R^{e})C(O)R^{f}, —S(O)NR^{e}R^{f}, —S(O)₂NR^{e}R^{f}, —S(O)R^{e}, —S(O)₂R^{e}, —NR^{e}R^{f}, —OR^{e}, —SR^{e}, C₁₋₆ alkyl, C₂₋₆ alkenyl and C₂₋₄ alkynyl; each R²⁰⁰ is independently selected from hydrogen, halogen, cyano, hydroxy, amino, oxo, thioxo, vinyl, —C(O)R^{e}, —C(O)OR³, —C(O)NR^{e}R^{f}, —N(R^{e})C(O)R^{f}, —S(O)NR^{e}R^{f}, —S(O)₂NR^{e}R^{f}, —S(O)R^{g}, —S(O)₂R^{g}, —NR^{e}R^{f}, —OR^{e}, —SR^{e}, C₁₋₆ alkyl, C₂₋₆ alkenyl and C₂₋₆ alkynyl; and, each R^{e} and R^{f} is independently selected from hydrogen, C₁₋₆ alkyl, C₂₋₆ alkenyl and C₂₋₆ alkynyl.

In some embodiments, the present disclosure provides compounds represented by structural Formula 2 and 2A:

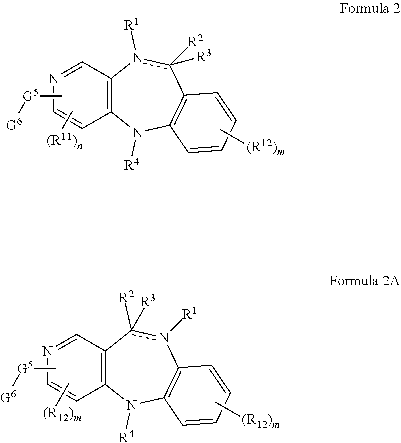

n is 0, 1 or 2;
m is 0, 1, 2, 3 or 4;

Each R¹¹ is independently selected from hydrogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{g}, —S(O)₂R^{g}, —NR^{a}R^{b}, —OR^{a}, —SR^{b}, C₁₋₆ alkyl, C₂₋₆ alkenyl, C₂₋₆ alkynyl, C3-8 cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocyclyl is optionally substituted with one to four R¹⁰⁰; alternatively, two R¹¹ groups together with the atoms to which they are attached to may form a cyclic group selected from C₃₋₈ cycloalkyl, C₆₋₁₀ aryl, 5-10 membered heteroaryl containing 1 to 4 heteroatoms selected from the group consisting of N, O, and S and 4-10 membered heterocyclyl containing 1 to 4 heteroatoms selected from the group consisting of N, O, and S;

Each R¹² is independently selected from hydrogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{g}, —S(O)₂R^{g}, —NR^{a}R^{b}, —OR^{a}, —SR^{b}, C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆alkyl, C₂₋₆ alkenyl, C₂₋₆ alkynyl, C₃₋₈ cycloalkyl, C₆₋₁₀ aryl, 5-10 membered heteroaryl and 4-10 membered heterocyclyl is optionally substituted with one to four R¹⁰⁰; alternatively, two R¹² groups together with the atoms to which they are attached to may form a cyclic group selected from C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S.

In some embodiments, the present disclosure provides compounds represented by structural Formula 3 and 3A:

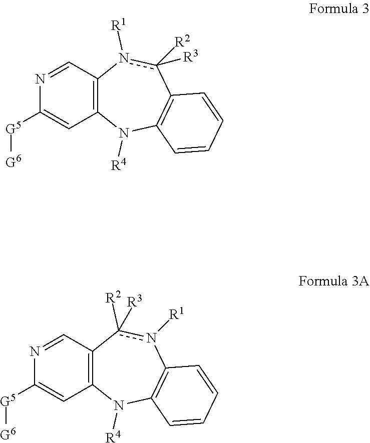

In some embodiments, the present disclosure provides compounds represented by structural

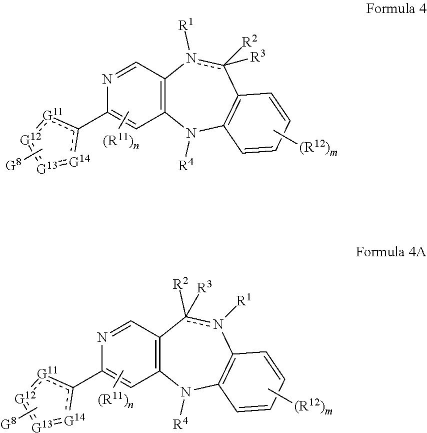

Each G¹¹, G¹², G¹³ and G¹⁴ is independently selected from N and CR¹⁰.

In some embodiments, the present disclosure provides compounds represented by structural Formula 5 and 5A:

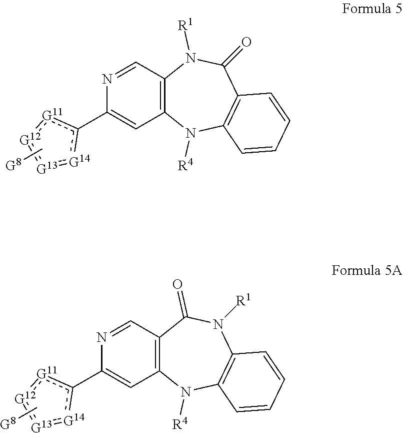

In some embodiments, a compound is utilized in accordance with the present disclosure in amorphous form. In some embodiments a compound is utilized in a crystalline form. For example, in some embodiments, a pharmaceutical composition comprising a compound of interest is prepared by processing and/or formulating compound in amorphous form. In some embodiments, a pharmaceutical composition comprising a compound of interest is prepared by processing and/or formulating compound in crystalline form. In some embodiments, a provided composition (e.g., a provided pharmaceutical composition) contains a compound of interesting amorphous form. Alternatively or additionally, in some embodiments, a provided composition (e.g., a provided pharmaceutical composition) contains a compound of interesting crystalline form.

In some embodiments, a provided composition is formulated for oral or intranasal delivery. Alternatively or additionally, in some embodiments, a provided composition is a solid composition.

The present invention also provides pharmaceutical compositions comprising one or more compounds of each of the formulae described herein or a pharmaceutically acceptable salt, tautomer, prodrug, solvate, metabolite, polymorph, analog or derivative thereof, and one or more pharmaceutically acceptable carriers.

The present invention also provides methods of treating a cell proliferative disorder by administering to a subject in need thereof, a therapeutically effective amount of a compound of each of the formulae described herein, or a pharmaceutically acceptable salt, tautomer, prodrug, solvate, metabolite, polymorph, analog or derivative thereof, in combination with a pharmaceutically acceptable carrier, such that the disorder is treated.

The present invention also provides methods of treating cancer by administering to a subject in need thereof, a therapeutically effective amount of a compound of each of the formulae described herein, or a pharmaceutically acceptable salt, tautomer, prodrug, solvate, metabolite, polymorph, analog or derivative thereof, in combination with a pharmaceutically acceptable carrier, such that the cancer is treated.

The present invention also provides methods of selectively inducing cell death in precancerous or cancerous cells by contacting a cell with an effective amount of a compound of each of the formulae described herein, or a pharmaceutically acceptable salt, tautomer, prodrug, solvate, metabolite, polymorph, analog or derivative thereof, in combination with a pharmaceutically acceptable carrier, such that contacting the cell results in selective induction of cell death in the precancerous or cancer cells.

The present invention also provides methods of treating neurodegenerative diseases or neurological disorders by contacting a cell with an effective amount of a compound of each of the formulae described herein, or a pharmaceutically acceptable salt, tautomer, prodrug, solvate, metabolite, polymorph, analog or derivative thereof, in combination with a pharmaceutically acceptable carrier, such that contacting the cell results treatment of neurodegenerative diseases or neurological disorders.

The present invention provides methods of synthesizing compounds of each of the formulae described herein, or pharmaceutically acceptable salts, tautomers, prodrugs, solvates, metabolites, polymorphs, analogs or derivatives thereof.

The present invention provides kits containing one or more compounds of each of the formulae described thereof or pharmaceutically acceptable salts, tautomers, prodrugs, solvates, metabolites, polymorphs, analogs or derivatives thereof. In one aspect, the present invention provides kits further containing a pharmaceutically active ingredient.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting.

A method is also disclosed herein for treating neurodegenerative diseases and cancer associated with one or more kinases or their mutants from the following list: AMPK-related kinases like NUAK1, NUAK2, SIK1, SIK2, SIK3, MARK1, MARK2, MARK3, MARK4; AURKA; AURKB; AURKC; CLK1; CLK2; DCAMKL2; MAPK7; MKNK2; PIK3CD; PKN3; RET; TAOK1; TAOK2; TAOK3 and ULK2, comprising administering to a subject in need thereof, a therapeutically effective amount of a compound of Formula I.

A composition (e.g., a pharmaceutical composition) is also disclosed comprising a compound as described herein and one or more pharmaceutically acceptable excipients. In some embodiments, provided herein is a method of inhibiting AMPK-related kinases like NUAK1, NUAK2, SIK1, SIK2, SIK3, MARK1, MARK2, MARK3, MARK4; AURKA; AURKB; AURKC; CLK1; CLK2; DCAMKL2; MAPK7; MKNK2; PIK3CD; PKN3; RET; TAOK1; TAOK2; TAOK3 and ULK2, with an effective amount of a compound or pharmaceutical composition as described herein.

In some embodiments, a method is provided for inhibiting one or more of these kinases wherein the said kinase is present in a cell. In other aspects, the inhibition can take place in a subject suffering from a disorder selected from various cancers, such as but not limited to, brain cancers, breast cancer, NSCLC, colorectal cancer, pancreatic cancer, and head and neck cancers. In other aspects, the inhibition can take place in a subject suffering from a neurodegenerative disease, neurological disorder or tauopathy. In other aspects the inhibition can take place in a subject suffering from inflammatory diseases. In some embodiments, a second therapeutic agent can be administered to the subject.

### DETAILED DESCRIPTION

### Definitions

As used herein, the following definitions apply to provided compounds, unless otherwise indicated. For purposes of this invention, chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Compounds described herein may be biologically or therapeutically active. Certain compounds described herein may be referred to as “pro-drugs,” “metabolites,” or “degradants.” Further, in some embodiments, one or more compounds described herein can be provided and/or utilized in any of a variety of forms such as, for example, crystal forms, salt forms, protected forms, ester forms, isomeric forms (e.g., optical and/or structural isomers), isotopic forms, etc. Those of skill in the art will appreciate that certain compounds have structures that can exist in one or more steroisomeric forms. In some embodiments, such compounds may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form. Those of skill in the art will appreciate that certain compounds have structures that can exist in one or more tautomeric forms. Those of skill in the art will appreciate that certain compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ³⁵C; ¹⁸F for ¹⁹F; ¹³¹I for ¹²⁷I; et). In some embodiments, such compounds may be utilized in accordance with the present disclosure in one or more isotopically modified forms, or mixtures thereof. In some embodiments, reference to a particular compound may relate to a specific form of that compound. In some embodiments, where a compound is one that exists or is found in nature, that compound may be provided and/or utilized in accordance in the present invention in a form different from that in which it exists or is found in nature. Those of ordinary skill in the art will appreciate that, in some embodiments, a compound preparation including a different level, amount, or ratio of one or more individual forms than a reference preparation or source (e.g., a natural source) of the compound may be considered to be a different form of the compound as described herein. Thus, in some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or mom atoms is a different isotope than is present in a reference preparation may also be considered to be a different form.

Further, as is known in the art, many chemical entities (in particular many organic molecules and/or many small molecules) can adopt a variety of different “solid forms” such as, for example, amorphous forms and/or crystalline forms (e.g., polymorphs, hydrates, solvates, etc). In some embodiments, such entities may be utilized as a single such form (e.g., as a pure preparation of a single polymorph). In some embodiments, such entities may be utilized as a mixture of such forms.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms (“C₁-C₆”). In some embodiments, aliphatic groups contain 1-5 aliphatic carbon atoms (“C₁-C₅”). In other embodiments, aliphatic groups contain 1-4 aliphatic carbon atoms (“C₁-C₄”). In still other embodiments, aliphatic groups contain 1-3 aliphatic carbon atoms (“C₁-C₃”), and in yet other embodiments, aliphatic groups contain 1-2 aliphatic carbon atoms (“C₁-C₂”). In some embodiments. “cycloaliphatic” (or “carbocycle” or “cycloalkyl”) refers to a monocyclic hydrocarbon containing 3-6 aliphatic carbon atoms (“C₃-C₆”) that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl,” as used herein, means an unbranched or branched chain, saturated, monovalent hydrocarbon residue containing 1 to 10 carbon atoms. Suitable alkyl groups include methyl, ethyl, n- and iso-propyl, n-, sec-, iso- and tert-butyl, neopentyl, and the like.

The term “alkenyl,” as used herein, means a monovalent straight or branched chain groups of, unless otherwise specified, from 2 to 10 carbon atoms (“C₂-C₁₀”) containing one or more carbon-carbon double bonds and is exemplified by ethenyl, propenyl, butenyl, pentenyl, hexenyl, and the like.

The term “alkynyl,” as used herein, means a monovalent straight or branched chain groups from 2 to 10 carbon atoms (“C₂-C₁₀”) containing at least one carbon-carbon triple bond. Suitable alkynyl groups include ethynyl, propynyl, butynyl, pentynyl, hexynyl, and the like.

The term “heteroatom,” as used herein, means one or more of oxygen, sulfur, nitrogen, phosphorus, or silicon (including any oxidized form of nitrogen, sulfur, phosphorus, or silicon; and the quaternized form of any basic nitrogen or a substitutable nitrogen of a heterocyclic ring.

The term “heteroalkyl,” as used herein, refers to an alkyl group, wherein one or more carbon atoms is replaced with a heteroatom selected from oxygen, sulfur, or nitrogen.

The term “unsaturated”, as used herein, means that a moiety has one or more units of unsaturation. In some embodiments, a unit of unsaturation is a carbon-carbon double bond (i.e., —C═C—). In some embodiments, a unit of unsaturation is a carbon-carbon triple bond (i.e., —C≡C—).

The term “bivalent C₂₋₈ (or C₂₋₆)unsaturated, straight or branched, hydrocarbon chain,” as used herein, means bivalent alkenylene and alkynylene chains that are straight or branched as defined herein and have one or more units of unsaturation.

The term “alkylene,” as used herein, means a straight or branched bivalent alkyl group. Exemplary alkylenes include —CH₂—, —CH₂CH₂—, —CH(CH₃)—, —CH₂CH(CH₃)—, —CH(CH₃)CH₂—, etc. In some embodiments, an “alkylene chain” is a polymethylene group, i.e., —(CH₂)ₙ—, wherein n is a positive integer, preferably from to 6, from 1 to 4, from 1 to 3, from 1 to 2, or from 2 to 3. A substituted alkylene chain is a bivalent alkyl group in which one or more hydrogen atoms are replaced with a substituent. Suitable substituents include those described below for a substituted aliphatic group.

The term “alkenylene,” as used herein, means a bivalent alkenyl group. A substituted alkenylene chain is a bivalent alkenyl group containing at least one double bond in which one or more hydrogen atoms are optionally replaced with a substituent. Suitable substituents include those described below for a substituted aliphatic group.

The term “halogen,” as used herein, means F, Cl, Br, or I.

The term “aryl,” as used herein, means monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteraryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14□ electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. When used in reference to a ring atom of a heteroryl, the term “nitrogen” includes a substituted nitrogen. As an example, in a heteroaryl ring having 0-3 heteroatoms selected from oxygen, sulfur or nitrogen, nitrogen may be N (as in pyridinyl—

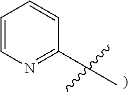 [SMILES: *c1ccccn1]

or ⁺NR\hat{ }(as in N-substituted pyridinyl—

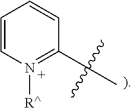

Heteroaryl groups may be mono- or bicyclic. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. When a heteroaryl ring is fused to an aryl ring, the term “heteroaro” is used to refer to the heteroaryl ring that is fused to the aryl ring. The term “heteroaryl” may be used interchangeably with the terms “heteroaryl ring”, “heteroaryl group”, or “heteroaromatic”, any of which terms include rings that are optionally substituted.

As used herein, the term “heterocycloalkyl” refer to a 4-7 membered cycloalkyl ring that is optionally substituted with one or more heteroatoms.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in piperidinyl

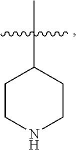 [SMILES: *C1CCNCC1]

3,4-dihydro-2H-pyrrolyl—

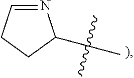 [SMILES: *C1CCC=N1]

NH (as in pyrrolidinyl—

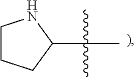 [SMILES: *C1CCCN1]

NR\hat{ } (as in N-substituted 2-pyrrolidinyl—

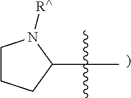

or ⁺NR\hat{ } (as in N-substituted 1-pyrrolidinyl—

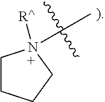

A heterocyclic ring can be attached to its pendant group at any heteroatom or carbon atom that results in a stable structure and any of the ring atoms can be optionally substituted. A heterocyclyl group may be mono- or bicyclic. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. For purposes of clarity, a “heterocyclic” ring includes a saturated or partially unsaturated ring having one or more heteroatoms, wherein the ring is either monocyclic or fused to one or more aryl, heteroaryl, or cycloaliphatic rings. When a heterocyclic ring is fused to an aryl ring, the term “heterocyclo” is used to refer to the heterocyclic ring that is fused to the aryl ring. A “saturated heterocyclic ring” refers to a saturated ring having one or more heteroatoms, wherein the ring is monocyclic or fused to one or more saturated cycloaliphatic rings.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, certain compounds of interest may contain one or more “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be independently halogen; —(CH₂)₀₋₄R^{o}; —(CH₂)₀₋₄OR^{o}; —O(CH)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄—N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —CH₂)₀₋₄C(O)OR; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}, SC(S)SR^{o}; —CH₂)₀₋₄SC(O)R; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}2; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), may be, independently, halogen, —(CH₂)₀₋₂ [symbol] , -(halo [symbol] ), —(CH₂)₀₋₂OH, —(CH₂)₀₋₂O [symbol] , —(CH₂)₀₋₂CH(O [symbol] )₂; —O(halo [symbol] ), —CN, —N₃, —(CH₂)₀₋₂C(O)) [symbol] , —(CH₂)₀₋₂C(O)OH, —(CH₂)₀₋₂C(O)O [symbol] , —(CH₂)₀₋₂S [symbol] , —(CH₂)₀₋₂SH, —(CH₂)₀₋₂NH₂, —(CH₂)₀₋₂NH [symbol] , —(CH₂)₀₋₂N [symbol] , —NO₂, —Si [symbol] , —OSi [symbol] ₃, —C(O)SR [symbol] , —(C₁₋₄ straight or branched alkylene)C(O)O [symbol] , or —SS [symbol] wherein each [symbol] is unsubstituted or where preceded by “halo” is substituted only with one or more halogens, and is independently selected from C₁₋₄ aliphatic, —CH₂P, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, NNHS(O)₂*, ═NR*, ═NOR*, —O(C(R*₂))₂₋₃O—, or —S(C(R*₂))₂₋₃S—, wherein each independent occurrence of R is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, — [symbol] , -(halo [symbol] ), —OH, —O [symbol] , —O(halo [symbol] ), —CN, —C(O)OH, —C(O)O [symbol] , —NH₂, —NH [symbol] , —N [symbol] ₂, or —NO₂, wherein each [symbol] is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{\}, —NR^{\}₂, —C(O)R^{\}, —C(O)OR^{\}, —C(O)C(O)R^{\}, —C(O)CH₂C(O)R^{\}, —S(O)₂R^{\}, —S(O)₂NR^{\}₂, —C(S)NR^{\}₂, —C(NH)NR^{\}, or —N(R^{\})S(O)₂R^{\}; wherein each R^{\} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{\}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{\} are independently halogen, — [symbol] , -(halo [symbol] ), —OH, —O [symbol] , —O(halo [symbol] ), —CN, —C(O)OH, —C(O)O [symbol] , —NH₂, —NH [symbol] , —N [symbol] ₂, or —NO₂, wherein each [symbol] is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{\} include ═O and ═S.

In some embodiments, structures depicted herein may include all isomeric (e.g., enantiomeric, diastereomeric, and geometric (or conformational)) forms of the depicted structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein may encompass compounds that differ from the depicted structure(s) only in the presence of one or more isotopically enriched atoms. For example, compounds having the presented structures including the replacement of hydrogen by deuterium or tritium, or the replacement of a carbon by a ³C- or ⁴C-enriched carbon are within the scope of this invention. Such compounds may be useful, for example, as analytical tools, as probes in biological assays, or as therapeutic agents in accordance with the present invention. In some embodiments, the R¹ group of formula 1 comprises one or more deuterium atoms.

Compounds provided herein are usually administered in the form of pharmaceutical compositions. Thus, provides herein are also pharmaceutical compositions that contain one or more of the compounds of any of the formulae disclosed herein or a pharmaceutically acceptable salt, isomers, prodrug, or solvate thereof, and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See. e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

Oral administration may be another route for administration of the compounds described herein. Administration may be via, for example, capsule or enteric coated tablets. In making the pharmaceutical compositions that include at least one compound of any of the formulae described herein or a pharmaceutically acceptable salt, prodrug, or solvate thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders. In certain embodiments, the pharmaceutical composition is in the form of tablets.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. 1621 Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose.

The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound of any of the formulae described herein or a pharmaceutically acceptable salt, isomer, prodrug, or solvate thereof, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound of any of the above formulae or a pharmaceutically acceptable salt, prodrug, or solvate thereof. When referring to these preformulation compositions as homogeneous, the active ingredient may be dispersed evenly throughout the composition so that the composition may be readily subdivided into equally effective unit dosage forms such as tablets, pills and capsules.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, diguconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N*(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. A pharmaceutical composition can also refer to a medicament.

As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

As used herein, a “prodrug”, is an entity that, when administered to an organism, is metabolized in the body to deliver a therapeutic agent of interest. Various forms of “prodrugs” are known in the art. For examples of such prodrug derivatives, see:
Design of Prodrugs, edited by H. Bundgaard, (Elsevier, 1985) and Methods in Enzymology, 42:309-396, edited by K. Widder, et al. (Academic Press, 1985);
A Textbook of Drug Design and Development, edited by Krogsgaard-Larsen;
Bundgaard, Chapter 5 “Design and Application of Prodrugs”, by H. Bundgaard, p. 113-191 (1991);
Bundgaard, Advanced Drug Delivery Reviews, 8:1-38 (1992);
Bundgaard, et al., Journal of Pharmaceutical Sciences, 77:285 (1988); and
Kakeya, et al., Chem. Pharm. Bull., 32:692 (1984).

### Other Definitions

As used herein, the term “administration” typically refers to the administration of a composition or compound to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration of a particular compound may be achieved by administration of a composition that includes or otherwise delivers the compound to the subject or system (or to a relevant part thereof or site therein). Thus, in some embodiments, administration of a compound may be achieved by administration of a composition comprising the compound. Alternatively or additionally, in some embodiments, administration of a compound may be achieved by administration of a composition that achieves delivery of the compound (e.g., of a composition that includes a prodrug or other variant of the compound that is metabolized to the compound upon administration of the composition).

As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “binding” typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agents or modalities to a subject receiving the other agents or modalities in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

As used herein, the terms “dosage form” or “unit dosage form” refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses am separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

As used herein, the term “inhibitor” refers to an agent, condition, or event whose presence, level, degree, type, or form correlates with decreased level or activity of another agent (i.e., the inhibited agent, or target). In general, an inhibitor may be or include an agent of any chemical class including, for example, small molecules, polypeptides, nucleic acids, carbohydrates, lipids, metals, and/or any other entity, condition or event that shows the relevant inhibitory activity. In some embodiments, an inhibitor may be direct (in which case it exerts its influence directly upon its target, for example by binding to the target); in some embodiments, an inhibitor may be indirect (in which case it exerts its influence by interacting with and/or otherwise altering a regulator of the target, so that level and/or activity of the target is reduced).

As used herein, the term “patient”, “subject”, or “test subject” refers to any organism to which provided compound or compounds described herein are administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.). In some embodiments, a subject may be suffering from, and/or susceptible to a disease, disorder, and/or condition (e.g., cancer, prostate-cancer, or castration-resistant prostate cancer.).

As used herein, the term “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, the term “response” with respect to a treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response to a cellular response, or to a tumor's response. Response may be measured according to a wide variety of criteria, including clinical criteria and objective criteria. Techniques for assessing response include, but are not limited to, assay assessment, clinical examination, positron emission tomatography, chest X-ray CT scan, MRI, ultrasound, endoscopy, laparoscopy, presence or level of tumor markers in a sample obtained from a subject, cytology, and/or histology. Regarding measuring tumor response, methods and guidelines for assessing response to treatment are discussed in Therasse et. al., “New guidelines to evaluate the response to treatment in solid tumors”, European Organization for Research and Treatment of Cancer, National Cancer Institute of the United States, National Cancer Institute of Canada, J. Natl. Cancer Inst., 2000, 92(3):205-216. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria

As used herein, a “therapeutic regimen” refers to a dosing regimen whose administration across a relevant population may be correlated with a desired or beneficial therapeutic outcome.

As used herein, a “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

As used herein, the term “treatment” (also “treat” or“treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### Methods

The application provides methods of treating a disease in a subject comprising administering to the subject a compound, pharmaceutically acceptable salt, ester or prodrug of formulae 1, 1A, 2, 2A, 3, 3A, 4, 4A, 5 and 5A.

In one aspect, the invention provides a method, where in the disease is mediated by a kinase selected from AMPK-related kinases such as NUAK1, NUAK2, SIK1, SIK2, SIK3, MARK1, MARK2, MARK3, MARK4, as well as AURKA, AURKB, AURKC, CLK1, CLK2, DCAMKL2, MAPK7, MKNK2, PIK3CD, PKN3, RET, TAOK1, TAOK2, TAOK3, ULK2 and their mutants. In a further aspect, the kinase is NUAK1, SIK2 or CLK1.

In another embodiment, the invention provides a method wherein the disease is a neurodegenerative disease or neurological disorder.

In a further embodiment, the disease is a tauopathy, including but not limited to Alzheimer's disease, Pick disease, Progressive supranuclear palsy, Corticobasal degeneration, Argyrophilic grain disease, Chronic traumatic encephalopathy, Globular glial tauopathy, Mild cognitive impairment, Frontotemporal dementia, Parkinson's disease and Huntington disease.

In another embodiment, the invention provides a method wherein the disease is a cancer or proliferative disease.

In a further embodiment, the disease is cancer of the lung, colon, breast, prostate, liver, pancreas, brain, kidney, ovaries, stomach, skin, bone, blood and lymph and includes gliomas, renal carcinomas, head and neck squamous cell carcinomas, leukemias, lymphomas, myelomas and solid tumors.

In another embodiment, the disease is a metabolic disorder, including but not limited to type-2 diabetes.

In another embodiment, exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to 2H (deuterium, D), 3H (tritium), 11C, 13C, 14C, 15N, 18F, 31P, 32P, 35S, 36Cl and 125I. Various isotopically labeled compounds of the present disclosure, for example those into which radioactive isotopes such as 3H, 13C and 14C are incorporated. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds of Formula I in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium. The compounds according to the present application may be used in combination with one or more additional therapeutic agents.

The therapeutic agents may be in the forms of compounds, antibodies, polypeptides, or polynucleotides. The therapeutic agent includes, but is not limited to, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof. In one embodiment, the application provides a product comprising a compound described herein and an additional therapeutic agent as a combined preparation for simultaneous, separate or sequential use in therapy, e.g. a method of treating a disease, disorder, or condition that is mediated by PI3K isoforms.

Therapeutic agents may be delivered to a diseased cell using targeting agents which are directly linked to the therapeutic agent, wherein the targeting agents includes, but is not limited to, antibodies, antibody fragments, peptides or proteins. Therapeutic agents may be delivered to a diseased cell using targeting agents which are indirectly linked to the therapeutic agent, as in the case of targeted carriers like liposomes and nanoparticles, wherein the targeting agent includes but is not limited to antibodies, antibody fragments, peptides or proteins. Typically, the cell binding agent (e.g., antibody) is covalently bound to the drug or the carrier by a linker.

Therapeutic agents may be in the form of degradation agent, wherein the therapeutic agent is modified through a linkage to a proteasome targeting agent.

Therapeutic agents that can be used in combination with compounds of the invention include enzalutamide, abiraterone, abiraterone acetate, apalutamide, galeterone, olaparib, niraparib, veliparib, rucaparib, flutamide, nilutamide, bicalutamide, ketonazole, orteronel, finasteride, dutasteride, bexlosteride, izonsteride, turosteride, episteride, dexamethasone, prednisone, leuprolide, goserelin, triptorelin, histrelin, estrogen, cyproterone acetate, spironolactone, flutamide, hydroxyflutamide, docetaxel, cabazitaxel, sipuleucel-T, ODM-201, VT-464, EPI-506, and combinations thereof.

Chemotherapeutic agents may be categorized by their mechanism of action into, for example, the following groups: anti-metabolites/anti-cancer agents, such as pyrimidine analogs (floxuridine, capecitabine, and cytarabine); purine analogs, folate antagonists and related inhibitors, antiproliferative/antimitotic agents including natural products such as vinca alkaloid (vinblastine, vincristine) and microtubule such as taxane (paclitaxel, docetaxel), driamycin, nocodazole, epothilones and navelbine, epidipodophyllotoxins (etoposide, teniposide); DNA damaging agents (actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, etoposide, triethylenethiophosphoramide); antibiotics such as dactinomycin (actinomycin D), daunorubicin, doxorubicin (driamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTlC); antiproliferative/antimitotic antimetabolites such as folic acid analogs (methotrexate); platinum coordination complexes (cisplatin, oxiloplatinim, carboplatin), procarbazine, hydroxyurca, mitotan, aminoglutethimide; hormones, hormone analogs (estrogen, tamoxifen, gosrelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives (tacrolimus, sirolimus azathioprine, mycophenolate); phytoestrogens (daidzein, glycitein, genisteinand growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (driamycin), daunorubicin, dactinomycin, eniposide, epirubicin, etoposide, idarubicin, irinotecan and mitoxantrone, topotecan, irinotecan, camptothesin), corticosteroids (cortisone, dexamethasone, hydrocortisone, methylprednisolone, prednisone, and prednisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin.

As used herein the term “chemotherapeutic agent” or “chemotherapeutic” (or “chemotherapy,” in the case of treatment with a chemotherapeutic agent) is meant to encompass any non-proteinaceous (i.e., non-peptidic) chemical compound useful in the treatment of cancer. Examples of chemotherapeutic agents include alkylating agents such as thiotepa and cyclophosphamide (CYTOXAN); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin I and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calichcamicin gammall and calicheamicin phill, see, e.g., Agnew, Chem. Intl. Ed. Engl, 33:83-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; PSK®; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (TAXOL® and docetaxel (TAXOTERE®); chlorambucil; gemcitabine (Gemzar®); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine®); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; FOLFIRI (fluorouracil, leucovorin, and irinotecan) and pharmaceutically acceptable salts, acids or derivatives of any of the above. One or more chemotherapeutic agent are used or included in the present application.

Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston®); inhibitors of the enzyme aromatase, which regulates estrogen production in the adrenal glands, such as, for example, 4(5)-imidazoles, aminoglutethimide, megestrol acetate (Megace®), exemestane, formestane, fadrozole, vorozole (Rivisor®), letrozole (Femara®), and anastrozole (Arimidex®); and anti-androgens such as flutamide, nilutamide, bicalutamide, leuprohde, and goserelin; and pharmaceutically acceptable salts, acids or derivatives of any of the above.

The anti-angiogenic agents include, but are not limited to, retinoid acid and derivatives thereof. 2-methoxyestradiol, ANGIOSTATIN®, ENDOSTATIN®, suramin, squalamine, tissue inhibitor of metalloproteinase-1, tissue inhibitor of metalloproternase-2, plasminogen activator inhibitor-1, plasminogen activator inhibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,I-3,4-dehydmproline, thiaproline, .alpha.-dipyridyl, beta-aminopropionitrile fumarate, 4-propyl-5-(4-pyridinyl)-2(3h)-oxazolone; methotrexate, mitoxantrone, heparin, interterons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpba-2-antiplasmin, bisantrene, lobenzarit disodium, n-2-carboxyphenyl-4-chloroanthronilic acid disodium or “CCA”, thalidomide; angiostatic steroid, carboxynaminoimidazole; metalloproteinase inhibitors such as BB94. Other anti-angiogenesis agents include antibodies, preferably monoclonal antibodies against these angiogenic growth factors: beta-FGF, alpha-FGF, FGF-5, VEGF isoforms, VEGF-C, HGF/SF and Ang-1/Ang-2. See Ferrara N. and Alitalo, K. “Clinical application of angiogenic growth factors and their inhibitors” (1999) Nature Medicine 5:1359-1364.

The anti-fibrotic agents include, but are not limited to, the compounds such as beta-aminoproprionitrile (BAPN), as well as the compounds disclosed in U.S. Pat. No. 4,965,288 to Palfreyman, et al., issued Oct. 23, 1990, entitled “Inhibitors of lysyl oxidase,” relating to inhibitors of lysyl oxidase and their use in the treatment of diseases and conditions associated with the abnormal deposition of collagen; U.S. Pat. No. 4,997,854 to Kagan, et al., issued Mar. 5, 1991, entitled “Anti-fibrotic agents and methods for inhibiting the activity of lysyl oxidase in situ using adjacently positioned diamine analogue substrate,” relating to compounds which inhibit LOX for the treatment of various pathological fibrotic states, which are herein incorporated by reference. Further exemplary inhibitors are described in U.S. Pat. No. 4,943,593 to Palfreyman, et al., issued Jul. 24, 1990, entitled “Inhibitors of lysyl oxidase,” relating to compounds such as 2-isobutyl-3-fluoro-, chloro-, or bromo-allylamine; as well as, e.g., U.S. Pat. Nos. 5,021,456; 5,5059,714; 5,120,764; 5,182,297; 5,252,608 (relating to 2-(1-naphthyloxymemyl)-3-fluoroallylamine); and U.S. Patent Application No. 2004/0248871, which are herein incorporated by reference. Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone. Also, the anti-fibrotic agents are copper chelating agents, penetrating or not penetrating the cells. Exemplary compounds include indirect inhibitors such compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, sodium-4-mercaptobutanesulphinate trihydrate.

The immunotherapeutic agents include and are not limited to therapeutic antibodies suitable for treating patients; such as abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, obinutuzumab, CC49 and 3F8. The exemplified therapeutic antibodies may be further labeled or combined with a radioisotope particle, such as indium In 111, yttrium Y 90, iodine I-131.

The application also provides method for treating a subject who is undergoing one or more standard therapies, such as chemotherapy, radiotherapy, immunotherapy, surgery, or combination thereof. Accordingly, one or more therapeutic agent or inhibitors may be administered before, during, or after administration of chemotherapy, radiotherapy, immunotherapy, surgery or combination thereof.

Other examples of chemotherapy treatments (including standard or experimental chemotherapies) are described below. In addition, treatment of certain lymphomas is reviewed in Cheson, B. D., Leonard, J. P., “Monoclonal Antibody Therapy for B-Cell Non-Hodgkin's Lymphoma” The New England Journal of Medicine 2008, 359(6), p. 613-626; and Wierda, W. G., “Current and Investigational Therapies for Patients with CLL” Hematology 2006, p. 285-294. Lymphoma incidence patterns in the United States is profiled in Morton, L. M., et al. “Lymphoma Incidence Patterns by WHO Subtype in the United States, 1992-2001” Blood 2006, 107(1), p. 265-276.

Examples of immunotherapeutic agents include, but are not limited to, rituximab (such as Rituxan), alemtuzumab (such as Campath MabCampath), anti-CD19 antibodies, anti-CD20 antibodies, anti-MN-14 antibodies, anti-TRAIL, Anti-TRAIL DR4 and DRS antibodies, anti-CD74 antibodies, apolizumab, bevacizumab. CHIR-12.12, epratuzumab (hLL2-anti-CD22 humanized antibody), galiximab, ha20, ibritumomab tiuxetan, lumiliximab, milatuzumab, ofatumumab, PRO131921, SGN-40, WT-1 analog peptide vaccine, WT 126-134 peptide vaccine, tositumomab, autologous human tumor-derived HSPPC-96, and veltuzumab. Additional immunotherapy agents includes using cancer vaccines based upon the genetic makeup of an individual patient's tumor, such as lymphoma vaccine example is GTOP-99 (MyVax®).

Examples of chemotherapy agents include adesleukin, alvocidib, antineoplaston AS2-1, antineoplaston A10, anti-thymocyte globulin, amifostine trihydrate, aminocamptothecin, arsenic trioxide, beta alethine, Bcl-2 family protein inhibitor ABT-263, ABT-199, BMS-345541, bortezomib (Velcade®), bryostatin 1, busulfan, carboplatin, campath-1H, CC-5103, carmustine, caspofungin acetate, clofarabine, cisplatin, Cladribine (Leustarin), Chlorambucil (Leukeran), Curcumin, cyclosporine, Cyclophosphamide (Cyloxan, Fndoxan, Endoxana, Cyclostin), cytarabine, denileukin diftitox, dexamethasone, DT PACE, docetaxel, dolastatin 10, Doxorubicin (Adriamycin@, Adriblastine), doxorubicin hydrochloride, enzastaurin, epoetin alfa, etoposide, Everolimus (RAD001), fenretinide, fllgrastim, melphalan, mesna, Flavopiridol, Fludarabine (Fludara), Geldanamycin (17-AAG), ifosfamide, irinotecan hydrochloride, ixabepilone, Lenalidomide (Revlimid®, CC-5013), lymphokine-activated killer cells, melphalan, methotrexate, mitoxantrone hydrochloride, motexafin gadolinium, mycophenolate mofetil, nearabine, oblimersen (Genasense) Obatoclax (GX15-070), oblimersen, octreotide acetate, omega-3 fatty acids, oxaliplatin, paclitaxel, PD0332991, PEGylated liposomal doxorubicin hydrochloride, pegfilgrastim, Pentstatin (Nipent), perifosine, Prednisolone, Prednisone, R-roscovitine (Selicilib, CYC202), recombinant interferon alfa, recombinant intereukin-12, recombinant interleukin-11, recombinant flt3 ligand, recombinant human thrombopoietin, rituximab, sargramostim, sildenafil citrate, simvastatin, sirolimus, Styryl sulphones, tacrolimus, tanespimycin, Temsirolimus (CC1-779), Thalidomide, therapeutic allogeneic lymphocytes, thiotepa, tipifarnib, Velcade® (bortezomib or PS-341), Vincristine (Oncovin), vincristine sulfate, vinorelbine ditartrate, Vorinostat (SAHA), vorinostat, and FR (fludarabine, rituximab), CHOP (cyclophosphamide, doxorubicin, vincristine, prednisone), CVP (cyclophosphamide, vincristine and prednisone), FCM (fludarabine, cyclophosphamide, mitoxantrone), FCR (fludarabine, cyclophosphamide, rituximab), hyperCVAD (hyperfractionated cyclophosphamide, vincristine, doxorubicin, dexamethasone, methotrexate, cytarabine), ICE (iphosphamide, carboplatin and etoposide), MCP (mitoxantrone, chlorambucil, and prednisolone), R-CHOP (rituximab plus CHOP), R-CVP (rituximab plus CVP), R-FCM (rituximab plus FCM), R-ICE (rituximab-ICE), and R-MCP (R-MCP).

The therapeutic treatments can be supplemented or combined with any of the abovementioned therapies with stem cell transplantation or treatment. One example of modified approach is radioimmunotherapy, wherein a monoclonal antibody is combined with a radioisotope particle, such as indium In 111, yttrium Y 90, iodine 1-131. Examples of combination therapies include, but are not limited to, lodine-131 tositumomab (Bexxar®), Yttrium-90 ibritumomab tiuxetan (Zevalin®), Bexxar® with CHOP.

Other therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, pharmacological study, low-LET cobalt-60 gamma ray therapy, bleomycin, conventional surgery, radiation therapy, and nonmyeloablative allogencic hematopoietic stem cell transplantation.

As inhibitors of protein kinases, the compounds and compositions of this invention are also useful in biological samples. One aspect of the invention relates to inhibiting protein kinase activity in a biological sample, which method comprises contacting said biological sample with a compound of the invention or a composition comprising said compound. The term “biological sample”, as used herein, means an in vitro or an ex vivo sample, including, without limitation, cell cultures or extracts thereof; biopsied material obtained from a mammal or extracts thereof: and blood, saliva, urine, feces, semen, tears, or other body fluids or extracts thereof. Inhibition of protein kinase activity in a biological sample is useful for a variety of purposes that are known to one of skill in the art. Examples of such purposes include, but are not limited to, blood transfusion, organ-transplantation, and biological specimen storage.

Another aspect of this invention relates to the study of protein kinases in biological and pathological phenomena; the study of intracellular signal transduction pathways mediated by such protein kinases; and the comparative evaluation of new protein kinase inhibitors. Examples of such uses include, but are not limited to, biological assays such as enzyme assays and cell-based assays, the use of the disclosed compounds as chemical inducers of dimerization and the use of the disclosed compounds as chemical probes.

The activity of the compounds as protein kinase inhibitors may be assayed in vitro, in vivo or in a cell line. In vitro assays include assays that determine inhibition of either the kinase activity or ATPase activity of the activated kinase. Alternate in vitro assays quantitate the ability of the inhibitor to bind to the protein kinase and may be measured either by radiolabeling the inhibitor prior to binding, isolating the inhibitor/kinase complex and determining the amount of radiolabel bound, or by running a competition experiment where new inhibitors are incubated with the kinase bound to known radioligands.

### Examples

The following non-limiting examples provide those of ordinary skill in the art with possible case scenarios and specific methods to treat conditions within the scope of the present disclosure and are not intended to limit the scope of the disclosure.

General Synthesis

Typical embodiments of compounds described herein may be synthesized using the general reaction schemes described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Given a desired product for which the substituent groups are defined, the necessary starting materials generally may be determined by inspection. Starting materials are typically obtained from commercial sources or synthesized using published methods. For synthesizing compounds which are embodiments described in the present disclosure, inspection of the structure of the compound to be synthesized will provide the identity of each substituent group. The identity of the final product will generally render apparent the identity of the necessary starting materials by a simple process of inspection, given the examples herein.

Synthetic Reaction Parameters

The terms “solvent”, “inert organic solvent”, or “inert solvent” refer to a solvent inert under the conditions of the reaction being described in conjunction therewith (including, for example, benzene, toluene, acetonitrile, tetrahydrofuran (“THF”), dimethylformamide (“DMF”), chloroform, methylene chloride (or dichloromethane), diethyl ether, methanol, and the like). Unless specified to the contrary, the solvents used in the reactions of the present invention are inert organic solvents, and the reactions are carried out under an inert gas, preferably nitrogen or argon.

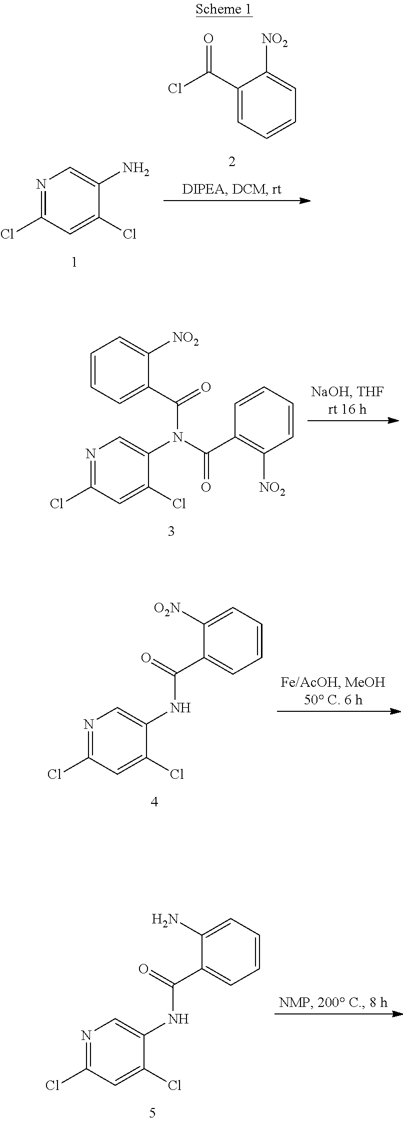 [SMILES: O=C(Cl)c1ccccc1[N+](=O)[O-] ; Nc1cnc(Cl)cc1Cl ; O=C(c1ccccc1[N+](=O)[O-])N(C(=O)c1ccccc1[N+](=O)[O-])c1cnc(Cl)cc1Cl ; O=C(Nc1cnc(Cl)cc1Cl)c1ccccc1[N+](=O)[O-] ; Nc1ccccc1C(=O)Nc1cnc(Cl)cc1Cl]

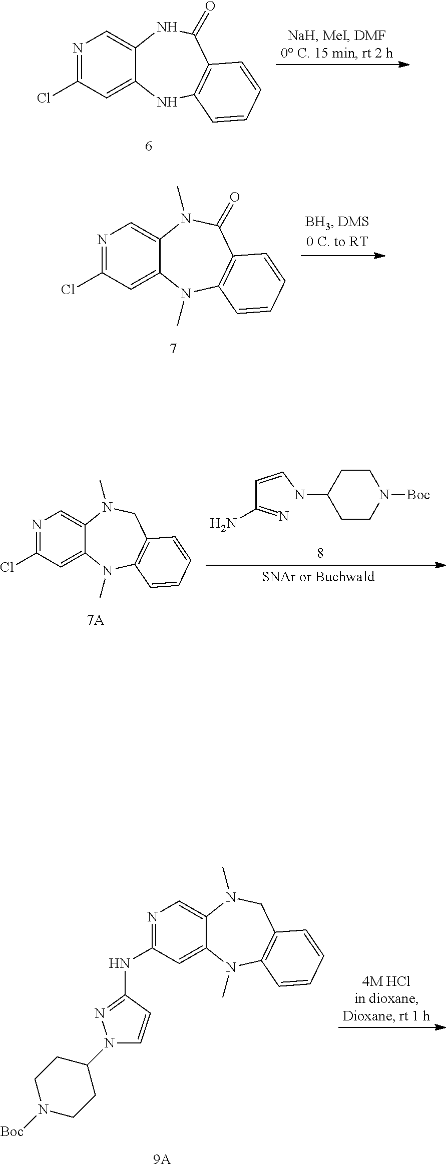 [SMILES: O=C1Nc2cnc(Cl)cc2Nc2ccccc21 ; CN1C(=O)c2ccccc2N(C)c2cc(Cl)ncc21 ; CN1Cc2ccccc2N(C)c2cc(Cl)ncc21 ; CC(C)(C)OC(=O)N1CCC(N/C=C\C(=N)N)CC1 ; CN1Cc2ccccc2N(C)c2cc(Nc3ccn(C4CCN(C(=O)OC(C)(C)C)CC4)n3)ncc21]

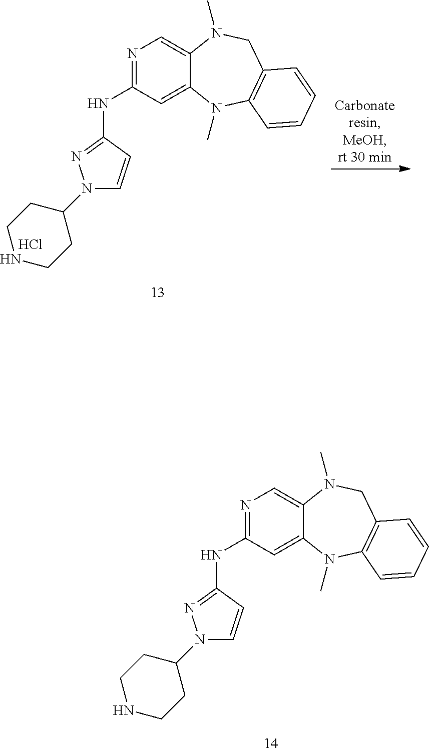

LCMS Conditions:

LCMS-Condition 01: Method:— LCMS_X-Select (Formic acid)

Column: X-Select CSI C18 (4.6*50) mm 2.5u, Mobile Phase: A.0.1% Formic acid in water B. 0.1% Formic acid in Acetonitrile Inj Volume; 5.0 μL, Flow Rate: 1.0, mL/minute, Gradient program: 2% B to 98% B in 2.8 minute, Hold till 4.8 min, At 5.0 min B conc is 2% up to 7.0 min.

LCMS-Condition 02: Method:— LCMS_X-Bridge (NH₃)

Column: X-Bridge C18 (3.0*50)mm 2.5μ; Mobile Phase: A. 0.05% NH₃ in water, B. 0.05% NH₃ in Acetonitrile Inj Volume; 0.2 μL, Flow Rate: 1.0 mL/minute; Gradient program:1% B to 90% B in 1.5 minute, 100% B IN2.5 minute, Hold till 2.8 minute, At 3.0 minute B conc is 1% up to 4.0 min Column: X-Select CSH C18 (3.0*50) mm 2.5u; Mobile Phase: A: 5 mM Ammonium Bicarbonate) in water; B: Acetonitrile; Inj Volume: 2 μL, Flow Rate: 1.2 mL/minute; LCMS-Condition 03: Method:— LCMS_X-Select (Ammonium Bicarbonate)

Column oven temp. 50 C; Gradient program: 0% B to 98% B in 2.0 minute, hold till 3.0 min, at 3.2 min B conc is 0% up to 4.0 min.

### Synthesis of N-(4,6-dichloropyridin-3-yl)-2-nitro-N-(2-nitrobenzoyl)benzamide (3)

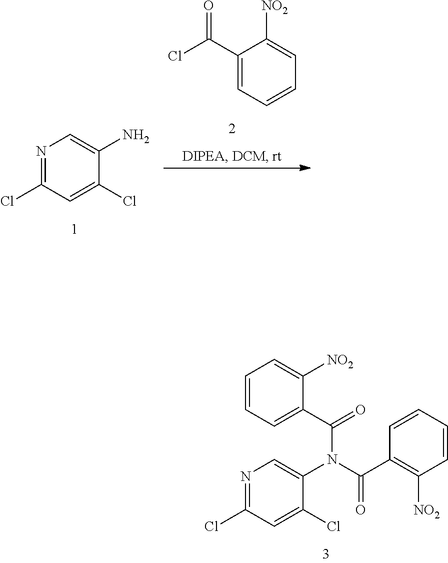 [SMILES: O=C(Cl)c1ccccc1[N+](=O)[O-] ; Nc1cnc(Cl)cc1Cl ; O=C(c1ccccc1[N+](=O)[O-])N(C(=O)c1ccccc1[N+](=O)[O-])c1cnc(Cl)cc1Cl]

To a stirred solution of 4,6-dichloropyridin-3-amine 1(5.00 g, 30.67 mmol) in DCM (80 mL) at 0° C. was added D1PEA (26.9 mL, 154.70 mmol) and 2-nitrobenzoyl chloride 2(14.0 g, 75.44 mmol). The reaction mixture was allowed to attain room temperature and stirred for 2 h. After completion of the reaction (monitored by TLC), the reaction mixture was quenched with water (50 mL) and extracted with ethyl acetate (2×50 mL).The combined organic layer was dried over anhydrous NaSO₄, filtered and concentrated under reduced pressure. The resulting crude compound was purified by silica gel column chromatography eluting with 100% DCM to afford 10 g(71% yield) of the title compound 3 as light brown solid.

LCMS-Condition-1: [M+H]⁺=461.40; Rt=1.962 min

¹H NMR (400 MHz, DMSO-d): 8.62 (s, 1H), 8.23 (d, J=8.31 Hz, 2H), 8.06 (s, 1H), 7.89 (d, J=7.34 Hz, 2H), 7.83-7.87 (m, 2H), 7.72-7.78 (m, 2H).

### Synthesis of N-(4,6-dichloropyridin-3-yl)-2-nitrobenzamide (4)

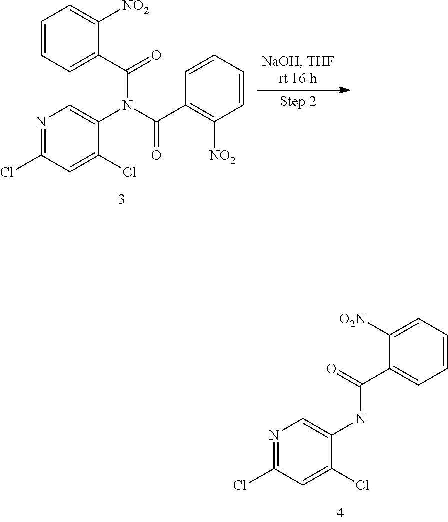 [SMILES: O=C(c1ccccc1[N+](=O)[O-])N(C(=O)c1ccccc1[N+](=O)[O-])c1cnc(Cl)cc1Cl ; O=C(Nc1cnc(Cl)cc1Cl)c1ccccc1[N+](=O)[O-]]

To a stirred solution of N-(4,6-dichloropyridin-3-yl)-2-nitro-N-(2-nitrobenzoyl)benzamide 3 (10.0 g 21.68 mmol) in THF (80 mL) was added aqueous solution of sodium hydroxide (1.30 g, 32.50 mmol) at room temperature and stirred for 16 h. After completion of the reaction (monitored by TLC), the reaction mixture was quenched with water (50 mL) and extracted with ethyl acetate (3×50 ml.). The combined organic layer was dried over anhydrous Na₂SO₄, filtered and concentrated under reduced pressure to afford 6.00 g (88% yield) of the title compound 4 as light brown solid.

LCMS-Condition-1: [M+H]⁺=311.95; Rt=1.797 min

¹H NMR (400 MHz, DMSO-d6) δ: 10.74 (s, 1H), 8.72 (s, 1H), 8.19 (d, J=7.82 Hz, 1H), 7.95 (s, 1H), 7.88-7.93 (m, 1H), 7.82 (d, J=2.93 Hz, 1H), 7.76-7.81 (m, 1H).

### Synthesis of 2-amino-N-(4,6-dichloropyridin-3-yl)benzamide (5)

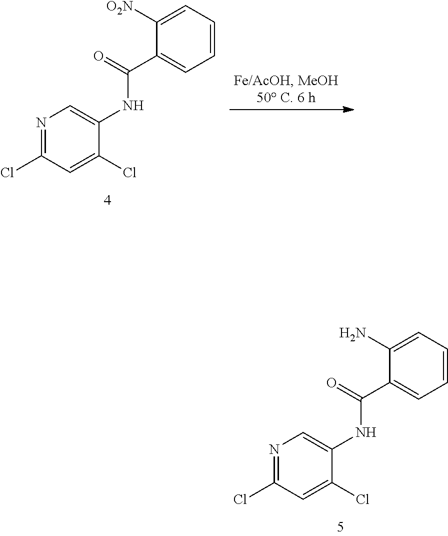 [SMILES: O=C(Nc1cnc(Cl)cc1Cl)c1ccccc1[N+](=O)[O-] ; Nc1ccccc1C(=O)Nc1cnc(Cl)cc1Cl]

To a stirred solution of N-(4,6-dichloropyridin-3-yl)-2-nitrobenzamide 4 (6.00 g, 19.22 mmol) in acetic acid: methanol (1:1; 60 mL) was added iron powder (4.00 g, 71.63 mmol) at room temperature. The reaction mixture was further heated to 50° C. for 6 h. After completion of the reaction (monitored by TLC), the unreacted iron powder was removed by filtration. The filtrate was quenched with aqueous sodium hydroxide solution followed by saturated solution of sodium chloride and extracted with ethyl acetate (3×50 mL). The combined organic layer was dried over anhydrous Na₂SO₄, filtered and concentrated under reduced pressure to afford 2.80 g (51% yield) of the title compound 5 as light brown solid. The compound was used for the next step without further purification.

LCMS-Condition-1: [M+H]⁺=282.10; Rt=1.744 min

¹H NMR (400 MHz, DMSO-d₆): 10.06 (br, s, 1H), 8.55 (s, 1H), 7.92 (s, 1H), 7.73 (d, J=7.82 Hz, 1H), 7.24 (t, J=7.83 Hz, 1H), 6.77 (d, J=8.31 Hz, 1H), 6.60 (t, J=7.34 Hz, 2H), 6.51 (br, s, 1H)

### Synthesis of 3-chloro-5,11-dihydro-10H-benzo[e]pyrido[3,4-b][1,4]diazepin-10-one (6)

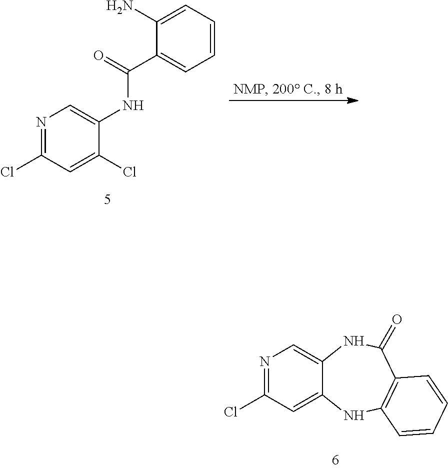 [SMILES: Nc1ccccc1C(=O)Nc1cnc(Cl)cc1Cl ; O=C1Nc2cnc(Cl)cc2Nc2ccccc21]

A solution of 2-amino-N-(4,6-dichloropyridin-3-yl)benzamide 5 (2.80 g, 9.925 mmol) in NMP (10 mL) was heated to 200° C. for 8 h. After completion of the reaction (monitored by TLC), the reaction mixture was quenched with water (100 mL) and the solid precipitated was filtered and dried under reduced pressure to afford 1.69 g (69% yield) of the title compound 6 as yellow solid. The compound was used for the next step without further purification.

LCMS-Condition-1: [M+H]⁺=245.90; Rt=1.646 min

¹H NMR (400 MHz, DMSO-d₆) δ: 10.03 (s, 1H), 8.65 (s, 1H), 7.85 (s, 1H), 7.75 (dd, J=1.47, 7.82 Hz, 1H), 7.36-7.42 (m, 1H), 6.95-6.97 (m, 1H), 6.94 (s, 2H).

### Synthesis of 3-chloro-5,11-dimethyl-5,11-dihydro-10H-benzo[e]pyrido[3,4-b][1,4]diazepin-10-one(7)

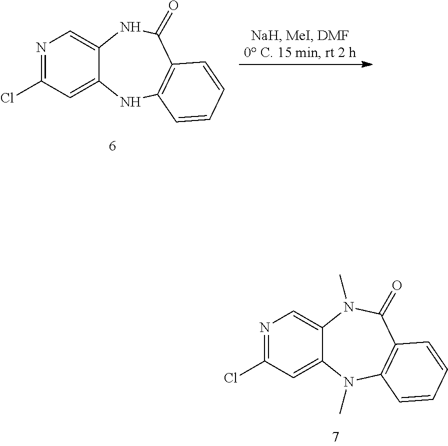 [SMILES: O=C1Nc2cnc(Cl)cc2Nc2ccccc21 ; CN1C(=O)c2ccccc2N(C)c2cc(Cl)ncc21]

To a stirred solution of 3-chloro-5,11-dihydro-0H-benzo[e]pyrido[3,4-b][1,4]diazepin-10-one 6 (1.20 g, 4.884 mmol) in DMF (15 mL) at 0° C. was added 60% dispersion of sodium hydride in oil (0.455 g, 11.37 mmol) followed by methyl iodide (0.73 mL, 11.69 mmol). The reaction mixture was allowed to attain room temperature and stirred for 2 h. After completion of the reaction (monitored by TLC), the reaction mixture was quenched with water (50 mL) and extracted with ethyl acetate (2×50 mL). The combined organic layer was dried over anhydrous Na₂SO₄, filtered and concentrated under reduced pressure to afford 1.20 g (90% yield) of 7 as light yellow solid.

LCMS-Condition-1: [M+H]⁺=274.05; Rt=1.890 min

¹H NMR (400 MHz, DMSO-d₆) δ: 8.36 (s, 1H), 7.65 (d, J=7.83 Hz, 1H), 7.49-7.54 (m, 1H), 7.34 (s, 1H), 7.21 (d, J=7.82 Hz, 1H), 7.16 (t, J=7.58 Hz, 1H), 3.45 (s, 3H), 3.31 (s, 3H).

### Synthesis of tert-butyl 4-(3-nitro-1H-pyrazol-1-yl)piperidine-1-carboxylate (12)

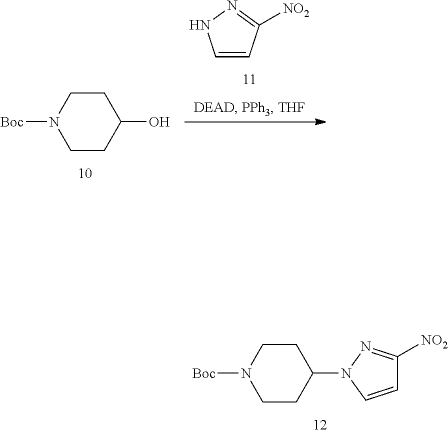 [SMILES: O=[N+]([O-])c1cc[nH]n1 ; CC(C)(C)OC(=O)N1CCC(O)CC1 ; CC(C)(C)OC(=O)N1CCC(n2ccc([N+](=O)[O-])n2)CC1]

To a stirred solution of tert-butyl 4-hydroxypiperidine-1-carboxylate 10 (8.90 g, 44.21 mmol) and 3-nitro-1H-pyrazole 11 (5.00 g, 44.21 mmol) in THF (80 ml) was added TPP (17.3 g, 65.99 mmol) and followed by dropwise addition of DEAD (10.3 mL, 65.97 mmol) at room temperature. The reaction mixture was further stirred at room temperature for 12 h. After completion of the reaction (monitored by TLC), the reaction mixture was quenched with water (50 mL) and extracted with ethyl acetate (2×50 mL). The combined organic layer was dried over anhydrous Na₂SO₄, filtered and concentrated under reduced pressure. The resulting crude compound was purified by silica gel column chromatography eluting with 0-30% ethyl acetate in n-hexane to afford 3.00 g (23% yield) of the title compound 12 as colorless oil.

Reaction was monitored by TLC(LC system: 50% ethyl acetate in n-hexane; R_{f}=0.4)

### Synthesis of ter-butyl 4-(3-amino-1H-pyrazol-1-yl)piperidine-1-carboxylate (8)

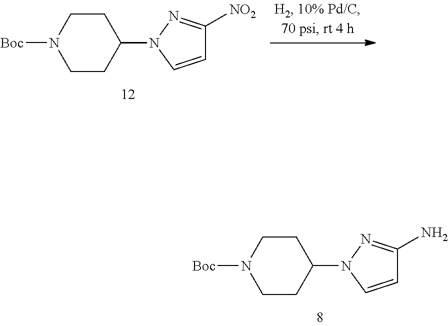 [SMILES: CC(C)(C)OC(=O)N1CCC(n2ccc([N+](=O)[O-])n2)CC1 ; CC(C)(C)OC(=O)N1CCC(n2ccc(N)n2)CC1]

To a stirred solution of tert-buty 4-(3-nitro-1H-pyrazol-t-yl)piperidine-1-carboxylate 12 (6.00 g, 20.25 mmol) in methanol (15 mL) was added 10% Pd/C (3.00 g, 50% wt of 12) at room temperature under argon atmosphere. The reaction mixture was further stirred under H₂ atmosphere 70 psi pressure at room temperature for 4 h. After completion of the reaction (monitored by TLC), the reaction mixture was filtered through a pad of Celite and the filtrate was concentrated under reduced pressure. The resulting crude compound was purified by silica gel column chromatography eluting with 0-3% methanol in DCM to afford 3.00 g (55% yield) of Int-8 as off white solid.

LCMS-Condition-1: [M+H]⁺=267.05; Rt=1.390 min

### Synthesis of 3-chloro-5,11-dimethyl-10,11-dihydro-5H-benzo[e]pyrido[3,4-b][1,4]diazepine (7A)

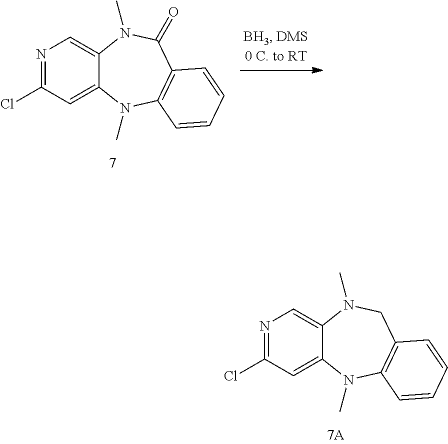 [SMILES: CN1C(=O)c2ccccc2N(C)c2cc(Cl)ncc21 ; CN1Cc2ccccc2N(C)c2cc(Cl)ncc21]

To a stirred solution of 3-chloro-5,11-dimethyl-5,11-dihydro-OH-benzo[c]pyrido[3,4-b][1,4]diazepin-10-one 7 (1.80 g, 6.576 mmol) in THE (15 mL) at 0° C. was added BH₃DMS (3.1 mL, 32.88 mmol). The reaction mixture was further heated to 70° C. for 1 h. After completion of the reaction (monitored by TLC), the reaction mixture was cooled to 0° C., quenched slowly under stirring with methanol and concentrated under reduced pressure upto dryness. The resulting crude compound was purified by silica gel column chromatography eluting with DCM to afford 1.20 g (70.6% yield) of the title compound 7A as thick yellow oil.

LCMS-Condition-1: [M+H]⁺=260.00; Rt=1.871 min

¹H NMR (400 MHz, DMSO-d₆) δ: 7.60 (s 1H), 7.33 (t, J=7.58 Hz, 1H), 7.26 (d, J=−7.34 Hz, 1H), 7.16 (d, J=7.83 Hz, 1H), 7.04 (t, J=7.34 Hz, 1H), 6.78 (s, 1H), 4.29 (s, 2H), 3.29 (s, 3H), 2.75 (s, 3H).

### Synthesis of tert-buty 4-(3-((5,11-dimethyl-10,11-dihydro-5H-benzo[e]pyrido[3,4-b][1,4]diazepin-3-yl)amino)-1H-pyrazol-1-yl)piperidine-1-carboxylate(9A)

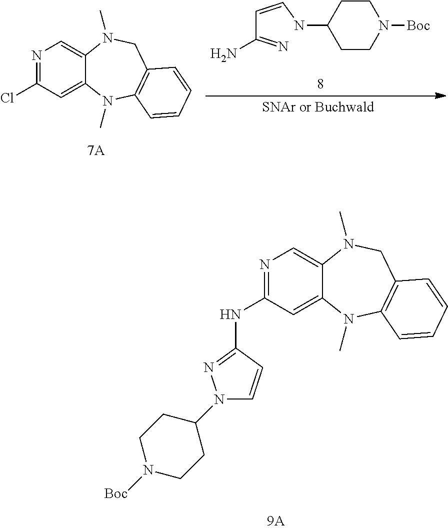 [SMILES: CN1Cc2ccccc2N(C)c2cc(Cl)ncc21 ; CC(C)(C)OC(=O)N1CCC(N/C=C\C(=N)N)CC1 ; CN1Cc2ccccc2N(C)c2cc(Nc3ccn(C4CCN(C(=O)OC(C)(C)C)CC4)n3)ncc21]

To 3-chloro-5,11-dimethyl-10,11-dihydro-5H-benzo[e]pyrido[3,4-b][1,4]diazepine 7A (0.600 g, 2.310 mmol) and tert-butyl 4-(3-amino-1H-pyrazol-1-yl)piperidine-1-carboxylate 8 (0.738 g, 2.772 mmol) and potassium tert-butoxide (0.777 g, 6.930 mmol) in dioxane (10 mL) was added X-phos (0.110 g, 0.231 mmol) and degassed with argon for 15 min. To the resulting solution was added catalyst Pd₂(dba)₃ (0.423 g, 0.462 mmol) and degassed with argon for another 2 min at room temperature. The reaction mixture was further heated at 100° C. for 1 h. After completion of the reaction (monitored by TLC), the reaction mixture was quenched with water (20 mL) and extracted with DCM (3×20 mL). The combined organic layer was dried over anhydrous Na₂SO₄, filtered and concentrated under reduced pressure. The resulting crude compound was purified by silica gel column chromatography eluting with 0-5% methanol in DCM to afford 0.160 g (14% yield) of the title compound 9A as a light brown solid.

LCMS-Condition-1: [M+H]⁺=490.25; Rt=1.617 min

¹H NMR (400 MHz, DMSO-d&) δ: 8.87 (br, s, 1H), 7.57 (d, J=2.93 Hz, 2H), 7.27-7.33 (m, 1H), 7.16-7.23 (m, 2H), 6.98-7.03 (m, 2H), 6.11 (d, J=1.96 Hz, 1H), 4.17-4.25 (m, 2H), 4.14 (s, 2H), 4.03 (d, J=10.76 Hz, 2H), 2.91 (d, J=4.89 Hz, 2H), 2.55 (s, 3H), 2.44-2.46 (m, 1H), 2.00 (d, J=10.76 Hz, 21), 1.74-1.84 (m, 3H),1.42 (s, 9H).

### Synthesis of 5,11-dimethyl-N-(1-piperidin-4-yl)-1H-pyrazol-3-yl)-10,11-dihydro-5H-benzo[e]pyrido[3,4-][1,4]diazepin-3-amine hydrochloride(13 HCl Salt)

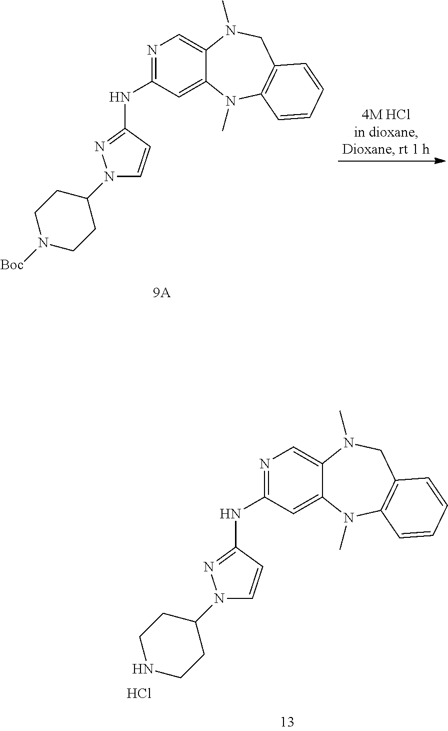 [SMILES: CN1Cc2ccccc2N(C)c2cc(Nc3ccn(C4CCN(C(=O)OC(C)(C)C)CC4)n3)ncc21 ; CN1Cc2ccccc2N(C)c2cc(Nc3ccn(C4CCNCC4)n3)ncc21 ; Cl]

To a stirred solution of tert-butyl 4-(3-((5,11-dimethyl-10,11-dihydro-5H1-benzo[e]pyrido[3,4-b][1,4]diazepin-3-yl)amino)-1H-pyrazol-1-yl)piperidine-1-carboxylate 9A (0.160 g, 0.326 mmol) in dioxane (1 mL) at 0° C. was added 4M HCl in dioxane (5 mL). The reaction mixture was allowed to attain room temperature and stirred for 30 min. After completion of the reaction (monitored by TLC), the reaction mixture was concentrated under reduced pressure resulting in the crude compound. The crude compound was purified by trituration in diethyl ether to afford 0.070 g (55% yield) of desired product 13 as HCl salt as white solid.

LCMS-Condition-1: [M+H]⁺=390.15; Rt=1.095 min

¹H NMR (400 MHz, DMSO-d6) δ: 12.87 (br, s, 1H), 10.93 (br, s, 1H), 9.32 (d, J=8.31 Hz, 1H), 8.93-9.02 (m, 1H), 7.85 (br, s, 1H), 7.56 (br, s, 1H), 7.37 (dd, J=7.83, 14.67 Hz, 2H), 7.15 (t, J=7.09 Hz, 1H), 6.75 (br, s, 1H), 5.99-6.04 (m, 1H), 4.45 (d, J=10.76 Hz, 1H), 4.31 (br, s, 2H), 3.40 (d, J=10.76 Hz, 2H), 3.00-3.12 (m, 2H), 2.72 (s. 3H) 2.56-2.64 (m, 3H), 2.40-2.45 (m, 2H), 2.20 (d, J=12.23 Hz, 21).

### Synthesis of 5,11-dimethyl-N-(1-(piperidin-4-yl)-1H-pyrazol-3-yl)-10,11-dihydro-5H-benzo[e]pyrido[3,4-b][1,4]diazepin-3-amine (14)

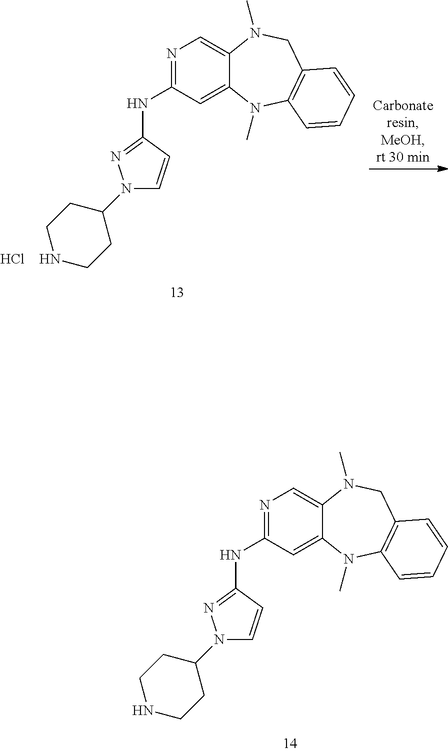 [SMILES: Cl ; CN1Cc2ccccc2N(C)c2cc(Nc3ccn(C4CCNCC4)n3)ncc21 ; CN1Cc2ccccc2N(C)c2cc(Nc3ccn(C4CCNCC4)n3)ncc21]

To a solution of 5,11-dimethyl-N-(1-(piperidin-4-yl)-1H-pyrazol-3-yl)-10,11-dihydro-5H1-benzo[e]pyrido[3,4-b][1,4]diazepin-3-amine hydrochloride 13 (0.045 g, 0.1155 mmol) in methanol (20 mL) at room temperature was added bicarbonate resin and stirred for 30 min. After completion of the reaction (monitored by TLC), the reaction mixture was filtered and concentrated under reduced pressure to afford 0.023 g (51.1% yield) free base of desired product 14 as white solid.

LCMS-Condition-1: [M+H]⁺=390.15; Rt=1.105 min

¹H NMR (400 MHz, DMSO-d) δ: 8.66 (br, s, 1H), 7.59 (s, 1H), 7.51 (br, s, 1H), 7.28 (d, J=6.85 Hz, 1H), 7.17-7.22 (m, 2H), 7.05 (br, s, 1H), 6.97-7.02 (m, 2H), 6.11 (br, s, 1H), 4.12 (br, s, 2H), 4.00 (d, J=11.251 Hz, 2H),3.03 (d, J=12.23 Hz, 2H), 2.53-2.57 (m, 6H), 1.93 (d, J=11.25 Hz, 2H),1.71-1.83 (m, 3H).

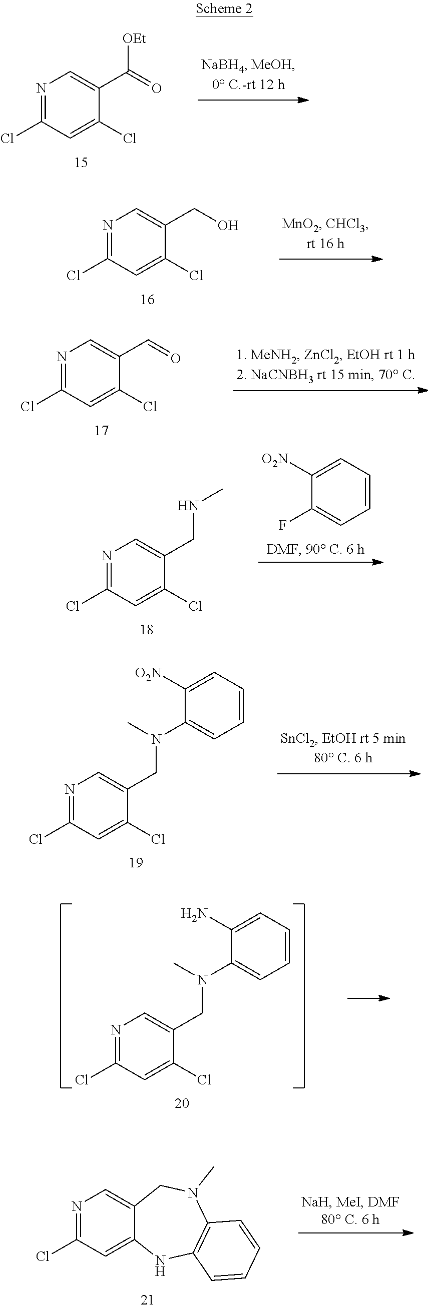

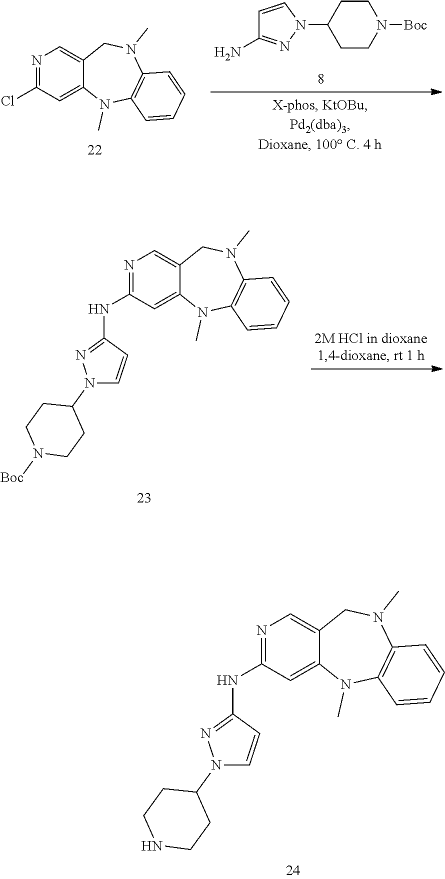 [SMILES: CN1Cc2cnc(Cl)cc2N(C)c2ccccc21 ; CC(C)(C)OC(=O)N1CCC(n2ccc(N)n2)CC1 ; CN1Cc2cnc(Nc3ccn(C4CCN(C(=O)OC(C)(C)C)CC4)n3)cc2N(C)c2ccccc21 ; CN1Cc2cnc(Nc3ccn(C4CCNCC4)n3)cc2N(C)c2ccccc21]

### Synthesis of (4,6-dichloropyridin-3-yl)methanol (16)

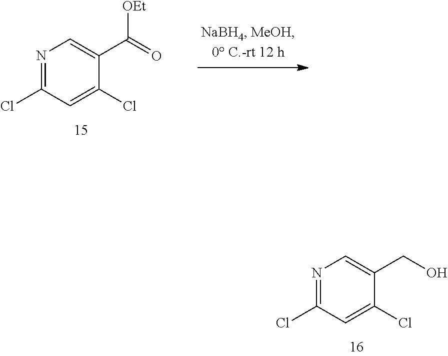 [SMILES: CCOC(=O)c1cnc(Cl)cc1Cl ; OCc1cnc(Cl)cc1Cl]

To a stirred solution of ethyl 4,6-dichloronicotinate 15 (5.00 g, 24.39 mmol) in methanol (50 mL) at 0° C. was added sodium borohydride (2.70 g, 73.17 mmol). The reaction mixture was allowed to attain room temperature and stirred for 12 h. After completion of the reaction (monitored by TLC), the reaction mixture was concentrated under reduced pressure, diluted with water (50 mL) and extracted with ethyl acetate (2×50 mL). The combined organic layer was dried over anhydrous Na₂SO₄, filtered and concentrated under reduced pressure to afford 4.00 g (93.0% yield) of the title compound 16 as white solid.

¹H NMR (400 MHz, DMSO-d6) δ: 8.46 (s, 1H), 7.76 (s, 1H), 5.59 (br, s, 1H), 4.59 (s, 2H).

### Synthesis of 4,6-dichloronicotinaldehyde (17)

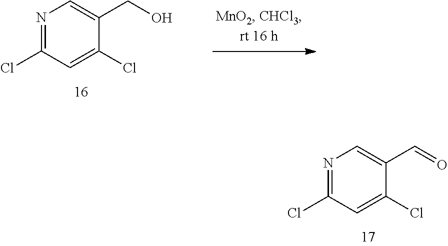 [SMILES: OCc1cnc(Cl)cc1Cl ; O=Cc1cnc(Cl)cc1Cl]

To a stirred solution of(4,6-dichloropyridin-3-yl)methanol 16 (8.00 g, 49.68 mmol) in CHCl₃ (80 mL) was added MnO₂ (43.0 g, 4%.8 mmol) at room temperature and stirred for 16 h. After completion of the reaction (monitored by TLC), the reaction mixture was filtered through a pad of Celite and the filtrate was concentrated under reduced pressure. The resulting crude compound was purified by 100-200 mesh size silica gel column chromatography eluting with 0-10% ethyl acetate in n-hexane to afford 3.80 g (44.2% yield) of the title compound 17 as grey solid.

¹H NMR (400 MHz, DMSO-d6): 10.25 (s, 1H), 8.81 (s, 1H), 8.03 (s, 1H).

### Synthesis of 1-(4,6-dichloropyridin-3-yl)-N-methylmethanamine (18)

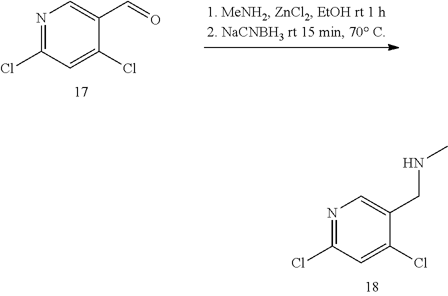

To a stirred solution of 4,6-dichloronicotinaldehyde 17 (3.80 g, 21.83 mmol) and methyl amine (4.84 mL, 109.2 mmol) in ethanol (30 mL) was added ZnCl (0.100 g, 0.733 mmol) and molecular sieves (0.100 g) at room temperature and stirred for 1 h. To the resulting solution was added NaCNBH₃ (5.50 g, 87.52 mmol) at room temperature and stirred for 15 min. The reaction mixture was further heated to 70° C. for 1 h. After completion of the reaction (monitored by TLC), the reaction mixture was diluted with water (mL) and extracted with DCM (3×50 mL). The combined organic layer was dried over anhydrous Na₂SO₄, filtered and concentrated under reduced pressure. The resulting crude compound was purified by 100-200 mesh size silica gel column chromatography eluting with 0-5% methanol in DCM to afford 2.40 g (58.5% yield) of the title compound 18 as colorless oil.

¹H NMR (400 MHz., DMSO-do) S: 8.44 (s, 1H), 7.75 (s, 1H), 3.73 (s, 2H), 2.29 (s, 31).

### Synthesis of N-((4,6-dichloropyridin-3-yl)methyl)-N-methyl-2-nitroaniline (19)

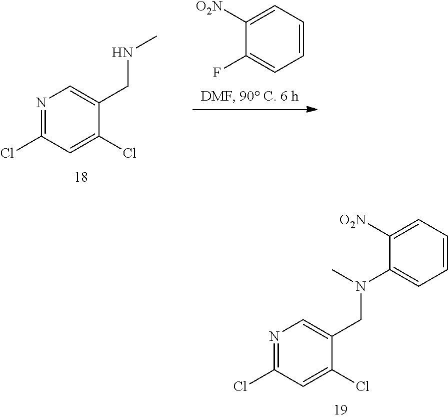 [SMILES: CNCc1cnc(Cl)cc1Cl ; O=[N+]([O-])c1ccccc1F ; CN(Cc1cnc(Cl)cc1Cl)c1ccccc1[N+](=O)[O-]]

To a solution of 1-(4,6-dichloropyridin-3-yl)-N-methylmethanamine 18 (2.40 g, 12.63 mmol) in DMF (5 mL) was added 1-fluoro-2-nitrobenzene (2.13 g, 15.15 mmol) at room temperature. The reaction mixture was further heated at 90° C. for 6 h. After completion of the reaction (monitored by TLC), the reaction mixture was diluted with water (100 mL) and extracted with DCM (2×50 mL). The combined organic layer was dried over anhydrous Na₂SO₄, filtered and concentrated under reduced pressure. The resulting crude compound was purified by 100-200 mesh size silica gel column chromatography eluting with 0-20% ethyl acetate in n-hexane to afford 2.20 g (56.4% yield) of the title compound 19 as yellow solid.

¹H NMR (400 MHz, DMSO-d₆) δ: 8.37 (s, 1-1), 7.81 (s, 1H), 7.76-7.80 (m, 1H), 7.50-7.56 (m, 1H), 7.33 (d. J=8.31 Hz, 1H), 7.04 (t, J=7.58 Hz, 1H), 4.43 (s, 2H), 2.75 (s, 3H).

### Synthesis of 3-chloro-10-methyl-10,11-dihydro-5H-benzo[b]pyrido[4,3-e][1,4]diazepine (21)

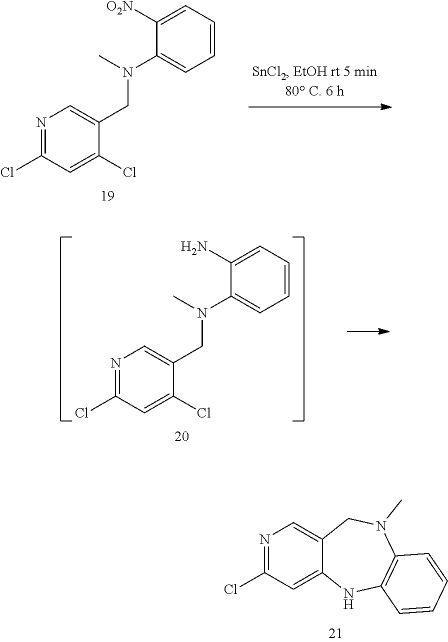

To a stirred solution of N(4,6-dichloropyridin-3-yl)methyl)-N-methyl-2-nitroaniline 19 (2.19 g. 7.041 mmol) in ethanol (20 mL) was added SnCl₂ (5.20 g, 28.16 mmol) at room temperature and stirred for 5 min. The reaction mixture was further heated at 80° C. for 6 h. After completion of the reaction (monitored by TLC), the reaction mixture was filtered through a pad of Celite and the filtrate was concentrated under reduced pressure upto dryness resulting in the crude residue. The residue was basified using IM NaOH solution and extracted with DCM (2×50 mL). The combined organic layer was dried over anhydrous Na₂SO₄, filtered and concentrated under reduced pressure to afford 1.50 g (88.2% yield) of the title compound 21 as off white solid.

¹H NMR (400 MHz, DMSO-d) δ: 9.18 (s, 1H), 7.78 (s, 1H), 6.90-7.00 (m, 2H), 6.82-6.89 (m, 2H), 6.80 (s, 1H), 3.96 (s, 2H), 2.86 (s, 3H).

### Synthesis of 3-chloro-5,10-dimethyl-10,11-dihydro-5H-benzo[b]pyrido[4,3-e][1,4]diazepine (22)

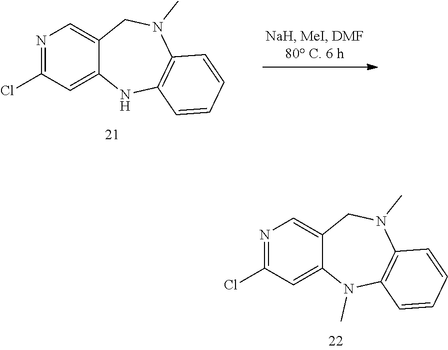 [SMILES: CN1Cc2cnc(Cl)cc2Nc2ccccc21 ; CN1Cc2cnc(Cl)cc2N(C)c2ccccc21]

To a stirred solution of 3-chloro-10-methyl-10,11-dihydro-H-benzo[b]pyrido[4,3-e][1,4]diazepine 21 (0.500 g, 2.040 mmol) in DMF (10 mL) at 0° C. was added 60% dispersion of sodium hydride in oil (0.163 g, 4.081 mmol) and stirred for 5 min. Followed by addition of methyl iodide (0.189 mL, 3.061 mmol) at the same temperature. The reaction mixture was further heated to 80° C. for 6 h. After completion of the reaction (monitored by TLC), the reaction mixture was diluted with ice cold water (50 mL) and stirred for 10 min. The solid precipitated was filtered and dried under vacuum to afford 0.500 g (94.5% yield) of 22 as light brown solid.

¹H NMR (400 MHz, DMSO-d₆) δ: 10.70 (s, 1H), 8.33 (s, 1H), 7.22 (s, 1H), 7.16 (d, J=7.48 Hz, 1H), 6.97-7.12 (m, 3H), 4.04 (s, 2H), 3.90 (s, 3H), 2.95 (s, 3H).

### Synthesis of tert-butyl 4-(3-((5,10-dimethyl-10,11-dihydro-5H-benzo[b]pyrido[4,3-e][1,4]diazepin-3-yl)amino)-1H-pyrazol-1-yl)piperidine-1-carboxylate (23)

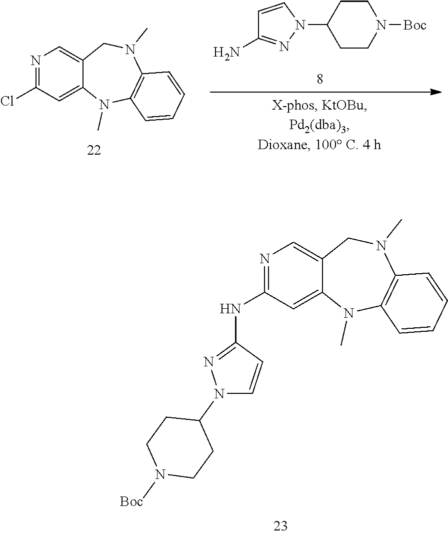 [SMILES: CN1Cc2cnc(Cl)cc2N(C)c2ccccc21 ; CC(C)(C)OC(=O)N1CCC(n2ccc(N)n2)CC1 ; CN1Cc2cnc(Nc3ccn(C4CCN(C(=O)OC(C)(C)C)CC4)n3)cc2N(C)c2ccccc21]

To a stirred solution of 3-chloro-5,10-dimethyl-10,11-dihydro-5H-benzo[b]pyrido[4,3-e][1,4]diazepine 22 (0.400 g, 1.540 mmol), tert-butyl 4-(3-amino-1H-pyrazol-1-yl)piperidine-1-carboxylate 8 (0.408 g, 1.531 mmol) and potassium tert-butoxide (0.518 g, 4.616 mmol) in dioxane (10 mL) was added X-phos (0.146 g, 0.306 mmol) and degassed with argon for 15 min. To the resulting solution was added catalyst Pd₂(dba)₃ (0.141 g, 0.154 mmol) and degassed with argon for another 10 min at room temperature. The reaction mixture was further heated at 100° C. for 4 h. After completion of the reaction (monitored by TLC), the reaction mixture was quenched with water (20 mL) and extracted with DCM (3×20 mL). The combined organic layer was dried over anhydrous Na₂SO₄, filtered and concentrated under reduced pressure. The resulting crude compound was purified by silica gel column chromatography eluting with 0-10% methanol in DCM to afford 0.140 g (18.5% yield) of the title compound 23 as light brown solid.

LCMS-Condition-1: [M+H]⁺=490.20; Rt=1.552 min.

### Synthesis of 5,10-dimethyl-N-(1-(piperidin-4-yl)-1H-pyrazol-3-yl)-10,11-dihydro-5H-benzo[b]pyrido[4,3-e][1,4]diazepin-3-amine (24)

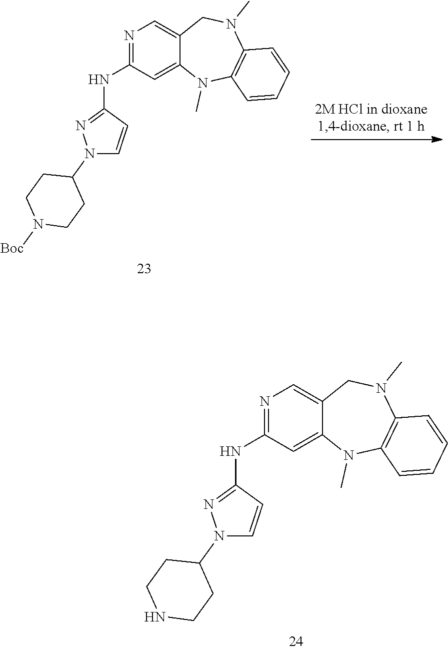 [SMILES: CN1Cc2cnc(Nc3ccn(C4CCN(C(=O)OC(C)(C)C)CC4)n3)cc2N(C)c2ccccc21 ; CN1Cc2cnc(Nc3ccn(C4CCNCC4)n3)cc2N(C)c2ccccc21]

To tert-butyl 4-(3-((5,10-dimethyl-10,11-dihydro-5H-benzo[b]pyrido[4,3-e][1,4]diazepin-3-yl)amino)-1H-pyrazol-1-yl)piperidine-1-carboxylate 23 (0.140 g, 0.286 mmol) in dioxane (5 mL) at 0° C. was added solution of 2M HCl in dioxane (1 mL). The reaction mixture was allowed to attain room temperature and stirred for 1 h. After completion of the reaction (monitored by TLC), the reaction mixture was concentrated under reduced pressure resulting in the crude compound. The resulting crude compound was purified by preparative HPLC purification to afford 0.020 g (17.9% yield) of 24 as yellow solid.

LCMS-Condition-1: [M+H]⁺=390.10; Rt=1.079 min

¹H NMR (400 MHz, CD₆OD) δ: 7.78 (s, 1H), 7.74 (d, J=2.49 Hz, 1H), 7.34 (d, J=7.98 Hz, 1H), 7.13-7.26 (m, 3H), 6.54 (s, 1H), 6.06 (d, J=2.49 Hz, 1H), 4.55 (ddd, J=5.49, 10.22, 15.21 Hz, 1H), 3.97 (s, 2H), 3.59 (s, 3H), 3.54-3.57 (m, 1H), 3.17-3.27 (m, 2H), 2.97 (s, 3H), 2.37 (br dd, J=3.99, 9.47 Hz, 4H).

### Synthesis of 5,10-Dimethyl-N-(4-(4-methylpiperazin-1-yl)phenyl)-10,11-dihydro-5H-benzo[b]pyrido[4,3-e][1,4]diazepin-3-amine (28)

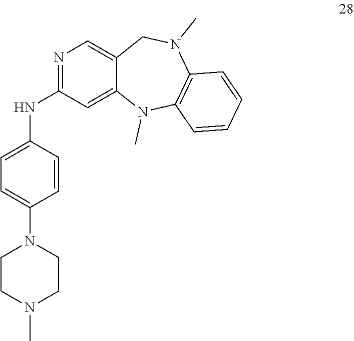 [SMILES: CN1CCN(c2ccc(Nc3cc4c(cn3)CN(C)c3ccccc3N4C)cc2)CC1]

General Synthetic Scheme:

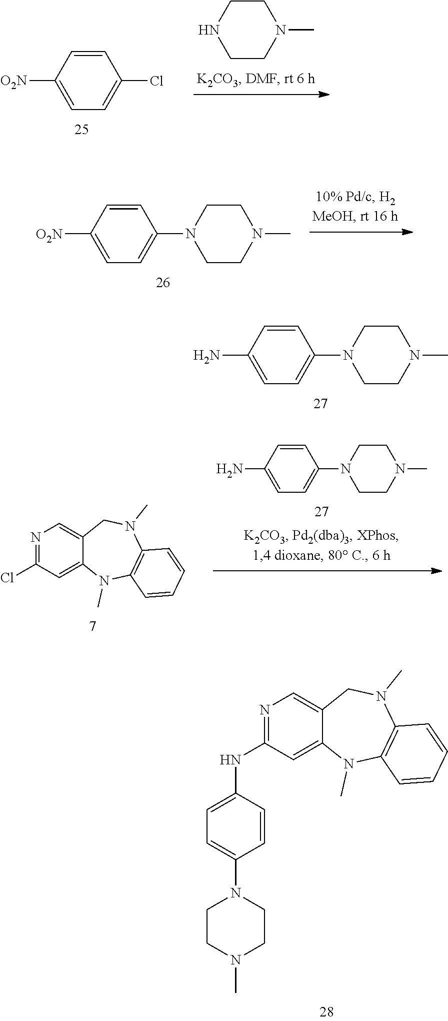

### Synthesis of 1-methyl-4-(4-nitrophenyl)piperazine (26)

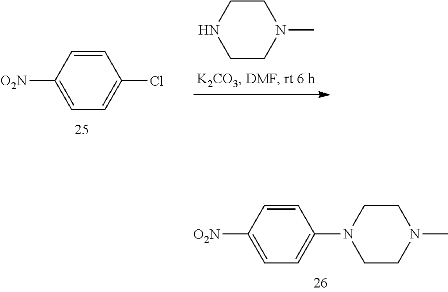

To a stirred solution of 1-chloro-4-nitrobenzene 25 (10.0 g, 63.47 mmol) in DMF (200 ml.) was added potassium carbonate (26.3 g, 191.1 mmol) and 1-methylpiperazine (9.55 g, 95.33 mmol) at room temperature and stirred at room temperature for 6 h. After completion of the reaction (monitored by TLC), the reaction mixture was quenched with water (50 mL) and solid precipitated was filtered and dried to afford 10.0 g (71.4% yield) of the title compound 26 as yellow solid.

¹H NMR (400 MHz, DMSO-d₆) δ: 8.04 (d, J=8.80 Hz, 2H), 7.02 (d, .=9.29 Hz, 2H), 3.41-3.47 (m, 4H), 2.39-2.44 (m, 4H), 2.21 (s, 3H).

### Synthesis of 4-(4-methylpiperazin-1-yl)aniline (27)

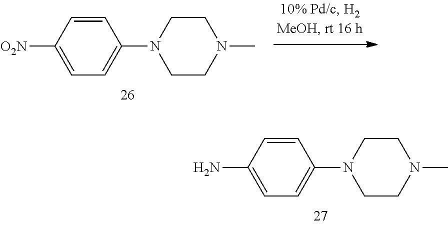 [SMILES: CN1CCN(c2ccc([N+](=O)[O-])cc2)CC1 ; CN1CCN(c2ccc(N)cc2)CC1]

To a stirred solution of 1-methyl-4-(4-nitrophenyl)piperazine 26 (10.0 g, 45.19 mmol) in methanol (100 mL) was added 10% Pd/C (3.00 g) at room temperature under argon atmosphere. The reaction mixture was further stirred under 112 atmosphere at room temperature for 16 h. After completion of the reaction (monitored by TLC), the reaction mixture was filtered through a pad of Celite and the filtrate was concentrated under reduced pressure to afford 6.50 g (75.6% yield) of 27 as grey solid.

¹H NMR (400 MHz, DMSO-d₆) δ: 6.81 (d, J=8.31 Hz, 2H), 6.62 (d, J=8.31 Hz, 2H), 4.69 (br, s, 2H), 2.99-3.07 (m, 4H), 2.64 (br, s, 2H), 2.52-2.59 (m, 4H), 2.34 (s, 3H).

### Synthesis of 5,10-dimethyl-N-(4-(4-methylpiperazin-1-yl)phenyl)-10,11-dihydro-5H-benzo[b]pyrido[4,3-e][1,4]diazepin-3-amine (28)

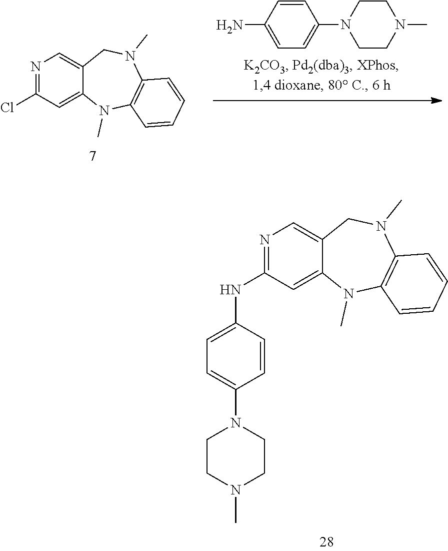 [SMILES: CN1Cc2cnc(Cl)cc2N(C)c2ccccc21 ; CN1CCN(c2ccc(N)cc2)CC1 ; CN1CCN(c2ccc(Nc3cc4c(cn3)CN(C)c3ccccc3N4C)cc2)CC1]

To 3-chloro-5,10-dimethyl-10,11-dihydro-5H-benzo[b]pyrido[4,3-e][1,4]diazepine 7 (0.100 g, 0.386 mmol) in dioxane (3 mL) was added potassium carbonate (0.106 g, 0.772 mmol), 4-(4-methylpiperazin-1-yl)aniline 27 (0.088 g, 0.463 mmol) at room temperature and degassed with argon for 20 min. To the resulting solution was added X-phos (0.018 g, 0.038 mmol) catalyst Pd(OAc)₂ (0.008 g, 0.038 mmol) at room temperature and degassed with argon for another 2 min. The reaction mixture was further heated at 80° C. for 6 h. After completion of the reaction (monitored by TLC), the reaction mixture was diluted with water (20 mL) and extracted with ethyl acetate (3×20 mL). The combined organic layer was dried over anhydrous Na₂SO₄, filtered and concentrated under reduced pressure. The resulting crude compound was purified by preparative HPLC purification to afford 0.022 g (13.8% yield) of 28 as grey solid.

LCMS-Condition-1: [M+H]⁺=415.20; Rt=1.070 min

¹H NMR (400 MHz, DMSO-d6) δ: 8.60 (br, s, 1H), 7.22 (s, 1H), 6.78-6.89 (m, 4H), 6.73 (td, J=6.17, 12.09 Hz, 3H), 6.63 (d, J=8.48 Hz, 2H), 3.70 (s, 2H), 2.99-3.04 (m, 4H), 2.79 (s, 3H), 2.46 (d, J=3.991 Hz, 4H), 2.22 (s, 3H)

¹H NMR (400 MHz, DMSO-d₆; D₂O exchange) δ 7.20 (br, s, 1H), 6.86 (d, J=7.98 Hz, 2H), 6.81 (d, J=3.49 Hz, 2H), 6.67-6.78 (m, 51), 3.69 (br, s, 2H), 3.34 (br, s, 3H), 2.97-3.05 (m, 4H), 2.75 (s, 3H), 2.41-2.47 (m, 4H), 2.17 (br, s, 3H).

### Synthesis of tert-butyl 4-(3-((5,11-dimethyl-10-oxo-10,11-dihydro-5H-benzo[e]pyrido[3,4-b][1,4]diazepin-3-yl)amino)-1H-pyrazol-1-yl)piperidine-1-carboxylate (29)

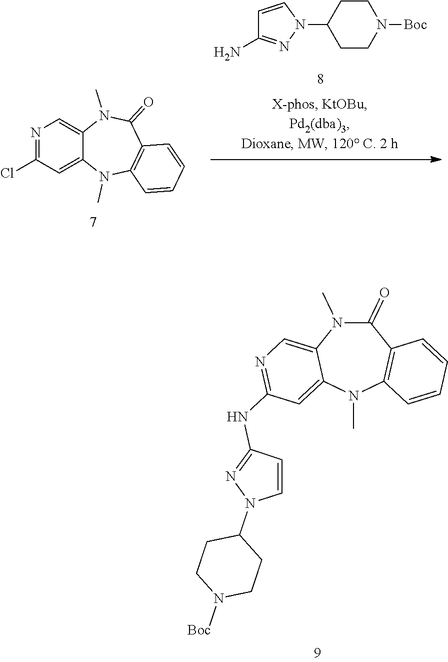 [SMILES: CC(C)(C)OC(=O)N1CCC(n2ccc(N)n2)CC1 ; CN1C(=O)c2ccccc2N(C)c2cc(Cl)ncc21 ; CN1C(=O)c2ccccc2N(C)c2cc(Nc3ccn(C4CCN(C(=O)OC(C)(C)C)CC4)n3)ncc21]

To a stirred solution of 3-chloro-5,11-dimethyl-5,11-dihydro-OH-benzo[e]pyrido[3,4-b][1,4]diazepin-10-one 7 (0.250 g, 0.915 mmol), tert-butyl 4-(3-amino-H-pyrazol-1-yl)piperidine-1-carboxylate 8 (0.292 g, 1.098 mmol) and potassium tert-butoxide (0.308 g, 2.745 mmol) in dioxane (4 mL) was added X-phos (0.043 g, 0.091 mmol) and degassed with argon for 15 min. To the resulting solution was added catalyst Pd₂(dba)₃ (0.167 g, 0.183 mmol) and degassed with argon for another 10 min at room temperature. The reaction mixture was further heated in microwave at 120° C. for 2 h. After completion of the reaction (monitored by TLC), the reaction mixture was quenched with water (20 mL) and extracted with DCM (3×20 mL). The combined organic layer was dried over anhydrous Na₂SO₄, filtered and concentrated under reduced pressure. The resulting crude compound was purified by silica gel column chromatography eluting with 0-3% methanol in DCM to afford 0.100 g (21.7% yield) of the title compound 29 as light brown solid.

LCMS-Condition-1: [M+H]⁺=504.30; Rt=1.721 min

### Synthesis of 5,11-dimethyl-3-((1-(piperidin-4-yl)-H-pyrazol-3-yl)amino)-5,11-dihydro-10H-benzo[e]pyrido[3,4-b][1,4]diazepin-10-one TFA Salt (30)

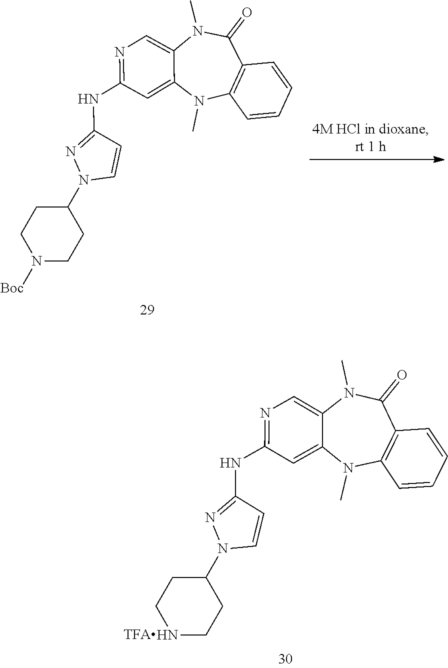 [SMILES: CN1C(=O)c2ccccc2N(C)c2cc(Nc3ccn(C4CCN(C(=O)OC(C)(C)C)CC4)n3)ncc21 ; CN1C(=O)c2ccccc2N(C)c2cc(Nc3ccn(C4CCNCC4)n3)ncc21]

To ter-buty 4-(3-((5,11-dimethyl-10-oxo-10,11-dihydro-5H-benzo[e]pyrido[3,4-b][1,4]diazepin-3-yl)amino)-1H-pyrazol-1-yl)piperidine-1-carboxylate 29 (0.099 g, 0.198 mmol) in dioxane (3 mL) at 0° C. was added 4M HCl in dioxane (mL). The reaction mixture was allowed to attain room temperature and stirred for 1 h. After completion of the reaction (monitored by TLC), the reaction mixture was concentrated under reduced pressure resulting in the crude compound. The resulting crude compound was purified by trituration in diethyl ether followed by preparative HPLC purification to afford 0.014 g (17.5% yield) of TFA salt 30 as white solid.

LCMS-Condition-1: [M+H]⁺=404.30; Rt=1.862 min

¹H NMR (400 MHz, DMSO-d) δ: 10.09 (br, s, 1H), 8.78 (br, s, 1H), 8.67 (br, s, 1H), 8.15 (s, 1H), 7.71 (d, J=2.45 Hz, 1H), 7.64-7.68 (in, 1H), 7.51 (t, J=7.09 Hz, 1H), 7.25 (d, J=8.31 Hz, 1H), 7.18 (br, s, 1H), 7.15-7.18 (m, 1H), 6.19 (d, J=1.96 Hz, 1H), 4.37-4.44 (m, 1H), 3.42 (s. 3H1), 3.39-3.41 (m, 2H),3.30 (s, 3H), 3.04-3.14 (m, 2H), 2.11-2.26 (m, 4H).

¹H NMR (400 MHz, CD₃OD) δ 8.20 (s, 1H), 7.79 (d, J=7.34 Hz, 1H), 7.76 (d, J=2.45 Hz, 1H), 7.54-7.60 (m, 1H), 7.30 (d, J=7.83 Hz, 1H), 7.25 (t, J=7.58 Hz, 1H), 6.92 (s, 1H), 6.14 (d, J=2.45 Hz, 1H), 4.51-4.58 (m, 1H), 3.58 (d, J=12.72 Hz, 2H), 3.54 (s, 3H), 3.46 (s, 3H), 3.18-3.26 (m, 4H), 2.34-2.41 (m, 4H).

| Example | Structure | Name |
|---|---|---|
| 1 | 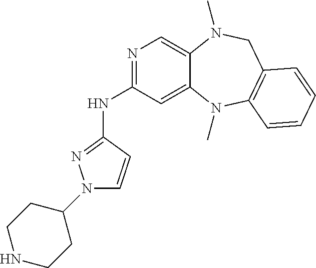 [SMILES: CN1Cc2ccccc2N(C)c2cc(Nc3ccn(C4CCNCC4)n3)ncc21] | 5,11-dimethyl-N-(1-(piperidin- 4-yl)-1H-pyrazol-3-yl)-10,11- dihydro-5H- benzo[e]pyrido[3,4- b][1,4]diazepin-3-amine |
| 2 | 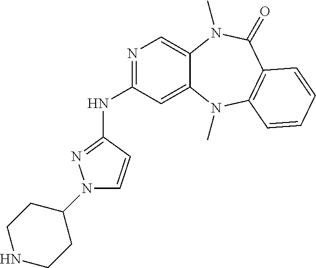 [SMILES: CN1C(=O)c2ccccc2N(C)c2cc(Nc3ccn(C4CCNCC4)n3)ncc21] | 5,11-dimethyl-3-((1-(piperidin- 4-yl)-1H-pyrazol-3-yl)amino)- 5,11-dihydro-10H- benzo[e]pyrido[3,4- b][1,4]diazepin-10-one |
| 3 | 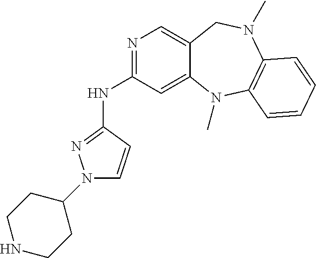 [SMILES: CN1Cc2cnc(Nc3ccn(C4CCNCC4)n3)cc2N(C)c2ccccc21] | 5,10-dimethyl-N-(1-(piperidin- 4-yl)-1H-pyrazol-3-yl)-10,11- dihydro-5H- benzo[b]pyrido[4,3- e][1,4]diazepin-3-amine |
| 4 | 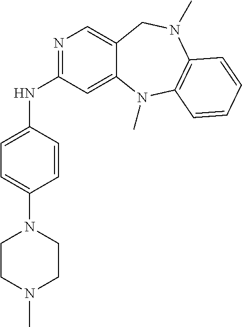 [SMILES: CN1CCN(c2ccc(Nc3cc4c(cn3)CN(C)c3ccccc3N4C)cc2)CC1] | 5,10-dimethyl-N-(4-(4- methylpiperazin-1-yl)phenyl)- 10,11-dihydro-5H- benzo[b]pyrido[4,3- e][1,4]diazepin-3-amine |

### In Vitro Kinase Inhibition Assays

Kinase activity and inhibition can be measured using binding assays as well as phosphorylation assays. In the binding assays, e.g. KinaseSeeker (Luceome Biotechnologies, Tucson, Ariz.) or Kinomescan (Eurofins), the competitive displacement of a kinase active site binding probe by a compound is detected and serves as a measure of compound inhibition. In phosphorylation assays, e.g. radioactivity assays or ADP-Glo (Promega) assays, the phosphorylation of a peptide or protein substrate is used to evaluate enzyme activity, and the reduction of phosphorylation in presence of a compounds serves as a measure of compound inhibition. The inhibitor compounds described herein are screened in the following manner.

In vitro kinase inhibition of disclosed compounds was evaluated at Luceome Biotechnologies (Tucson, Ariz.) using KinaseSeeker assays. Briefly, for the KinaseSeeker binding assays, stock solutions of compounds at 10 mM were prepared in DMSO. For kinase assays, a 24 uL aliquot of lysate containing Cfluc-kinase and Fos-Nfluc was incubated with either 1 uL of DMSO (for no-inhibitor control) or compound solution in DMSO (10 uM final compound concentration), for 2 hours in presence of a kinase specific probe. 80 uL of luciferin assay reagent was added to each solution and luminescence was immediately measured on a luminometer.

For phosphorylation assays, 30 ng of the active kinase (SignalChem) in kinase assay buffer was incubated with either inhibitor in DMSO or DMSO (no inhibitor control). Reaction was started by the addition of 10 mM magnesium sulfate, 100 uM [γ-³²P]-ATP and appropriate concentration of the relevant peptide substrate. After 3 hr incubation, the reaction was terminated by spotting 25 μl of the reaction mixture onto P81 paper strips. The paper strips were air dried, washed three times with 0.85% phosphoric acid and finally with acetone. The P81 paper was immersed in 10 mL of scintillation cocktail, and the radioactive counts were measured. For dose dependent curves, kinases were treated at varying concentrations of the compound in activity assays and IC50 values were determined using a four-parameter fit. The IC50 values against a select set of kinases is given below.

| | IC50 (micromolar) | | |
|---|---|---|---|
| | 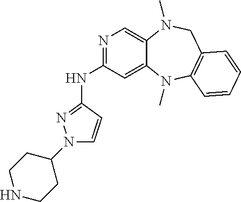 [SMILES: CN1Cc2ccccc2N(C)c2cc(Nc3ccn(C4CCNCC4)n3)ncc21] | 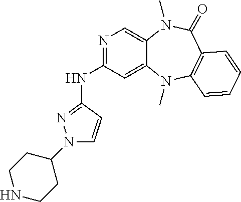 [SMILES: CN1C(=O)c2ccccc2N(C)c2cc(Nc3ccn(C4CCNCC4)n3)ncc21] | 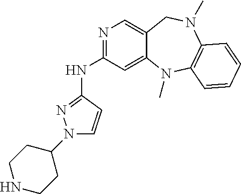 [SMILES: CN1Cc2cnc(Nc3ccn(C4CCNCC4)n3)cc2N(C)c2ccccc21] |
| Kinase | 14 | 30 | 24 |
| NUAK1 | 1.3 | 0.13 | 1.3 |
| NUAK2 | 4.6 | 0.34 | 7.0 |
| SIK2 | 18.6 | 1.5 | 0.69 |
| CLK1 | 2.7 | 57.8 | >50 |
| CLK2 | 1.5 | 33 | 9.5 |

### Example 4 In Vitro Kinase Selectivity Assays GP-6

Kinase selectivity was evaluated using the KinaseSeeker assay (Luceome Biotechnologies, Tucson, Ariz.). Briefly, stock solutions of compounds at 10 mM were prepared in DMSO. The compounds were screened against a panel of 411 kinases in duplicate at a final compound concentration of 10 uM. For kinase assays, a 24 uL aliquot of lysate containing Cfluc-kinase and Fos-Nfluc was incubated with either 1 uL of DMSO (for no-inhibitor control) or compound solution in DMSO, for 2 hours in presence of a kinase specific probe. 80 uL of luciferin assay reagent was added to each solution and luminescence was immediately measured on a luminometer.

Selectivity in KinaseSeeker assays was represented as Selectivity Score of S₅₀ (10 uM); where S₅₀ (10 uM) is the fraction of total kinases having less than 50% activity remaining upon treatment with 10 uM compound concentration. ZAN-2101 had a S₅₀ (10 uM) value of 0.017, ZAN-2102 had a S₅₀ (10 uM) of 0.078, and ZAN-2113 had a S₅₀ (10 uM) of 0.034. Selectivity data for ZAN-2101, ZAN-2102 and ZAN-2113 at 10 uM against 411 kinases is shown as a histogram.

### Example 5 Target Engagement Assays in Cells

Target engagement assays were carried out at Luceome Biotechnologies (Tucson. Ariz.) using Cellular KinaseSecker assays.

### Example 6 XTT Cell Viability Assay

Typical sources for cells include, but are not limited to, human bone marrow or peripheral blood lymphocytes, fibroblasts, tumors, immortalized cell lines, in-vitro transformed cell lines, rodent spleen cells, or their equivalents. Tumor cells and transformed cell lines are available from standard cell banks such as The American Type Culture Collection (ATCC). Cells genetically manipulated to express a particular kinase or kinases are also suitable for use in assaying cellular activity and can be made using standard molecular biology methods. These cells are grown in various standard tissue culture media available from suppliers such as GIBCO/BRL (Grand Island, N.Y.) supplemented with fetal bovine serum. Cellular activity may also be measured using bacterial, yeast, or virally infected mammalian cells. Standard inhibitors (or reference compounds) of cellular activities measured in cellular assays, include staurosporine (LC labs), wortmannin (Calbiochem), etc.

Cytotoxicity of compounds was evaluated indifferent cell lines including 120S and HepG2. Cryopreserved cells were passaged using standard protocols. U2OS was grown in McCoy's 5A supplemented with 10% fetal bovine serum, while HepG2 was grown in Minimum Essential Medium supplemented with 10% fetal bovine serum. Cells were plated in 96-well plates at cell densities of 2,000 cells/well and 5,000 cells/well and incubated at 37 deg C. and 5% CO2 in appropriate media. For the dose response studies, compounds were prepared in DMSO and added to the cells such that the final concentration of DMSO did not exceed 1%. Cells were evaluated 24, 48, and 72 hrs post-dosing for viability using XTT assay. XTT reagent was and added to the cells and the 96-well plate was incubated at 37 degC/5% CO2 for 2 hours, following which absorbance was measured at 450 nm. Compounds were tested in triplicate using staurosporine as a reference compound. The test compounds showed no cell death at the highest concentration tested (50 uM) at 24 hours for U2OS and HepG2 cells.

What is claimed is:

1. A compound of formula: 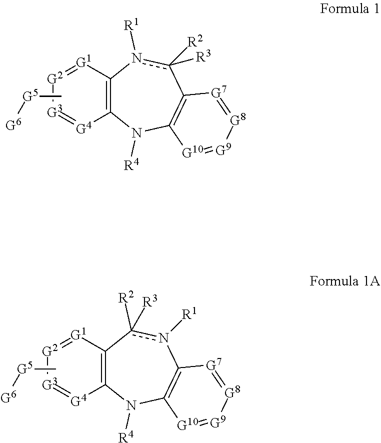
Each G¹, G², G³, G⁴, G⁷, G⁸, G⁹ and G¹⁰ is independently selected from N and CR¹⁰;
G⁵ is selected from —S—, —O—, —NR^{a}—, —C(O)—, —C(O)O—, —C(O)NR^{a}—, S(O)NR^{a}—, —S(O)₂NR^{a}—, —S(O)—, —S(O)₂—, C₁₋₆ alkylene, C₂₋₆ alkenylene and C₂₋₆ alkynylene; wherein each C₁₋₆ alkylene, C₂₋₆ alkenylene and C₂₋₆ alkynylene is substituted with one to six R¹⁰⁰;
G⁶ is selected from C₄₋₇ membered heterocycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₆₋₁₀ aryl, 5-10 membered heteroaryl and 4-10 membered heterocyclyl is optionally substituted with one to four R¹⁰⁰;
R¹ is selected from absent, hydrogen, cyano, hydroxy, amino, —C(O)R^{a}, —C(O)OR^{b}, —C(O)NR^{b}, —N(R^{a})C(O)R^{a}, —S(O)NR^{a}R^{b}, —S(O)₂NR^{a}R^{b}, —S(O)R^{a}, —S(O)₂R^{a}, — NR^{a}R^{b}, —OR^{a}, —SR^{b}, C₁₋₆ alkyl, C_{2- 6} alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S and 4-10 membered heterocyclyl containing 1 to 4 heteroatoms selected from the group consisting of N, O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocycly is optionally substituted with one to four R¹⁰⁰;
R² is selected from absent, hydrogen, halogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{a}, —S(O)₂R^{a}, —NR^{a}R^{b}, —OR^{a}, —SR^{b}, C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocycly is optionally substituted with one to four R¹⁰⁰;
R³ is selected from hydrogen, halogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{b}, —S(O)₂R^{g}, —NR^{a}R^{b}, —OR^{a}, —SR^{b}, C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocyclyl is optionally substituted with one to four R¹⁰⁰; and optionally R² and R³ together with the carbon atom to which it is attached may form a group selected from —C(O) and —C═CR^{a}R^{b};
R⁴ is selected from hydrogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{g}, —S(O)₂R^{g}, —NR^{a}R^{b}, —OR^{a}, —SR^{b}, C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocycly containing 1 to 4 heteroatoms selected from the group consisting of N, O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocyclyl is optionally substituted with one to four R¹⁰⁰;
Each R¹⁰ is independently selected from hydrogen, cyano, hydroxy, amino, —C(O)R^{a}, —C(O)OR^{b}, —C(O)NR^{b}, —N(R^{a})C(O)R^{b}, —S(O)NR^{a}R^{b}, —S(O)₂NR^{a}R^{b}, —S(O)R^{b}, —S(O)₂R^{g}, —NR^{a}R^{b}, —OR^{a}, —SR^{b}, C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocyclyl is optionally substituted with one to four R¹⁰⁰;
Each R^{a} and R^{b} is independently selected from hydrogen, C₁₋₆ alkyl, C₂₋₆ alkenyl and C₂₋₆ alkynyl;
wherein each CI-6 alkyl, C₂₋₆ alkenyl, C₂₋₆ alkynyl, is optionally substituted with one to four R²⁰⁰;
Each R^{c} and R^{d} is independently selected from hydrogen, C₆₋₁₀ aryl, C₁₋₆ alkyl, C₂₋₆ alkenyl and C₂₋₆ alkynyl;
Each R^{e} and R^{f} is independently selected from hydrogen, C₁₋₆ alkyl, C₂₋₆ alkenyl and C₂₋₆ alkynyl;
Each R^{g} is independently selected from C₁₋₆ alkyl, C₂₋₆ alkenyl, C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆alkyl, C₂₋₆ alkenyl, C₂₋₆alkynyl, C₃₋₈ cycloalkyl, C₆₋₁₀ aryl, 5-10 membered heteroaryl and 4-10 membered heterocyclyl is optionally substituted with one to four R²⁰⁰; each R¹⁰⁰ is independently selected from hydrogen, halogen, cyano, hydroxy, amino, oxo, thioxo, vinyl, —C(O)R^{c}, —C(O)OR^{c}, —C(O)NR^{c}R^{d}, —N(R^{c})C(O)R^{d}, —S(O)NR^{c}R^{d}, —S(O)₂NR^{c}R^{d}, —S(O)R^{c}, —S(O)₂R^{c}, NR^{c}R^{d}, —OR^{c}, —SR^{c}, C₁₋₆ alkyl, C₂₋₆ alkenyl and C₂₋₆ alkynyl, C₃₋₈ cycloalkyl, C₆₋₁₀ aryl, 5-10 membered heteroaryl containing 1 to 4 heteroatoms selected from the group consisting of N, O, and S, and 4-10 membered heterocyclyl containing 1 to 4 heteroatoms selected from the group consisting of N, O, and S; wherein each C₁₋₆ alkyl, C₂₋₆ alkenyl, C₂₋₆ alkynyl, C₃₋₈ cycloalkyl, C₆₋₁₀ aryl, 5-10 membered heteroaryl and 4-10 membered heterocyclyl is optionally substituted with one to four R²⁰¹; each R^{e} and R^{f} is independently selected from hydrogen, C₁₋₆ alkyl, C₂₋₆ alkenyl and C₂₋₆ alkynyl; each R²⁰¹ is independently selected from hydrogen, halogen, cyano, hydroxy, amino, oxo, thioxo, vinyl, —C(O)R^{e}, —C(O)OR^{e}, —C(O)NR^{e}R^{f}, —N(R^{e})C(O)R^{f}, —S(O)NR^{e}R^{f}, —S(O)₂NR^{e}R^{f}, —S(O)R^{e}, —S(O)₂R^{e}, —NR^{e}R^{f}, —OR^{e}, —SR^{e}, C₁₋₆ alkyl, C₂₋₄ alkenyl and C₂₋₆ alkynyl; each R²⁰⁰ is independently selected from hydrogen, halogen, cyano, hydroxy, amino, oxo, thioxo, vinyl, —C(O)R^{e}, —C(O)OR^{e}, —C(O)NR^{e}R^{f}, —N(R^{e})C(O)R^{f}, —S(O)NR^{e}R^{f}, —S(O)₂NR^{e}R^{f}, —S(O)R^{g}, —S(O)₂R^{g}, —NR^{e}R^{f}, —OR^{e}, —SR^{e}, C₁₋₆ alkyl, C₂₋₆ alkenyl and C₂₋₆ alkynyl; and, each R^{e} and R^{f} is independently selected from hydrogen, C₁₋₆ alkyl, C₂₋₆ alkenyl and C₂₋₆ alkynyl or a pharmaceutically acceptable salt, isomer, or a mixture thereof.
